(19) United States
(12) Patent Application Publication
(10) Pub. No.: US 2016/0199294 A1
(43) Pub. Date: Jul. 14, 2016

(54) SUBLINGUAL NALOXONE SPRAY

(71) Applicant: Insys Development Company, Inc., Chandler, AZ (US)

(72) Inventors: Kiran Amancha, Chandler, AZ (US); Shivani Chilampalli, Phoenix, AZ (US); Thrimoorthy Potta, Phoenix, AZ (US); Ning Shan, Chandler, AZ (US); Ningxin Yan, Chandler, AZ (US); Onkar N. Singh, Chandler, AZ (US); Venkat R. Goskonda, Phoenix, AZ (US)

(21) Appl. No.: 15/076,080

(22) Filed: Mar. 21, 2016

Related U.S. Application Data
(63) Continuation-in-part of application No. 14/730,585, filed on Jun. 4, 2015.
(60) Provisional application No. 62/022,041, filed on Jul. 8, 2014.

(51) Int. Cl. A61K 9/00; A61K 47/18; A61K 31/485

(52) U.S. Cl. CPC A61K 9/006; A61K 31/485; A61K 47/183

(57) ABSTRACT

The invention provides storage stable sublingual formulations containing naloxone, a pharmaceutically acceptable salt or a derivative thereof. The invention further provides methods for treating opioid dependence, opioid overdose, and congenital insensitivity to pain with anhidrosis by administering the sublingual formulations of the present invention to a patient in need thereof.

### FIELD OF THE INVENTION

The invention is directed to sublingual spray formulations containing naloxone, a pharmaceutically acceptable salt thereof, or a derivative thereof. The invention is further directed to methods of treating opioid dependence, opioid overdose, and congenital insensitivity to pain with anhidrosis by administering sublingual spray formulations containing naloxone, pharmaceutically acceptable salts thereof, or derivatives thereof to a patient in need thereof.

### BACKGROUND OF THE INVENTION

Naloxone has the following structure and is synthesized from thebaine:

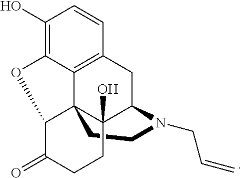

Naloxone is most commonly used to treat patients suffering from opioid dependence or overdose because it is a competitive μ-opioid antagonist that blocks the effects of opioids. Naloxone is currently available in Suboxone® (Suboxone is a registered trademark of Reckitt Benckiser Healthcare (UK) Limited) as tablet or sublingual film strip formulations. Suboxone® contains buprenorphine and naloxone in a 4:1 ratio.

One issue with other opioid dependence treatments is that they can become addictive. Naloxone, however, does not appear to be addictive and patients do not build up a tolerance.

Naloxone has also been used as a treatment for cognitive insensitivity to pain with anhidrosis. Insensitivity to pain with cognitive anhidrosis is a disorder in which the patient cannot feel pain.

Naloxone may be administered orally, intravenously, by injection or via the nasal mucosa. Naloxone has a low mean serum half-life when administered parentally. The quick metabolism may require repeat dosing or cause patient discomfort between doses. Enteral administration has low bioavailability due to hepatic first pass metabolism.

Accordingly, while there are some naloxone formulations currently available, there is a need for safe and effective sublingual spray formulations that are storage stable including physically and chemically stable and contain naloxone, pharmaceutically acceptable salts or a derivative thereof.

### SUMMARY OF THE INVENTION

In one aspect, the invention is directed to storage stable sublingual spray formulations comprising an effective amount of naloxone, a pharmaceutically acceptable salt thereof, or a derivative thereof, water, a cosolvent and a permeation enhancer.

In one aspect, the invention is directed to storage sublingual spray formulations comprising an effective amount of naloxone, a pharmaceutically acceptable salt or a derivative thereof, water, propylene glycol and ethanol as co-solvents, antioxidant, chelating agent and a permeation enhancer.

In another aspect, the invention is directed to storage stable sublingual spray formulations comprising an effective amount of naloxone, a pharmaceutically acceptable salt or a derivative thereof, water and a permeation enhancer that do not contain alcohol.

In yet another aspect, the invention is directed to methods for treating opioid dependence comprising administering the sublingual spray formulations of the present invention to a patient in need of opioid dependence treatment.

In a further aspect, the invention is directed to methods for treating opioid overdose comprising administering the sublingual spray formulations of the present invention to a patient in need of opioid overdose treatment.

In an additional aspect, the invention is directed to methods for treating congenital insensitivity to pain with anhidrosis comprising administering the sublingual spray formulations of the present invention to a patient in need of treatment for congenital insensitivity to pain with anhidrosis.

### BRIEF DESCRIPTION OF THE FIGURES

FIG. 1. Mean plasma concentration of Formulations #9A, #9A repeat #8A, #8AF and #7AF normalized to a 4 mg dosage. Values based on a geometric mean.

### DETAILED DESCRIPTION OF THE INVENTION

Applicants have created new sublingual naloxone formulations, which have high bioavailability and are storage stable, and have excellent droplet distribution when administered. Applicant created stable formulations with and without alcohol (see Examples 1, 4, 5 and 9). The formulations that do not contain alcohol are especially suitable for administration to children. Further, the alcohol-free formulations may be suitable for patients in recovery from alcohol addiction.

In a preferred embodiment, the sublingual naloxone formulation is a spray. In a more preferred embodiment the sublingual naloxone formulation is in liquid form. In yet a more preferred embodiment, the sublingual naloxone formulation is in a simple solution form. As used herein, the term “simple solution” refers to a solution in which the solute(s) has fully dissolved in the solvent.

As used herein, the term “storage stable” includes but is not limited physical and chemical stability.

Formulations with Alcohol

In one embodiment, the invention is directed to sublingual spray formulations comprising an effective amount of naloxone, a pharmaceutically acceptable salt or a derivative thereof, water as a solvent, a cosolvent and an antioxidant. In a preferred embodiment, naloxone is in salt form.

In another embodiment, the invention is directed to sublingual spray formulations comprising an effective amount of naloxone, a pharmaceutically acceptable salt or a derivative thereof, water as a solvent, a cosolvent and a permeation enhancer. In a preferred embodiment, naloxone is in salt form.

The cosolvent may be an alcohol, a glycol, or a mixture thereof. The formulations preferably contain from about 5 to about 90% w/w cosolvent. More preferably the formulations contain from about 20% to about 70% or from about 40% to about 65% w/w or from about 45% to about 55% w/w cosolvent. In a preferred embodiment, the formulations contain about 50% w/w or about 55% w/w cosolvent. In a most preferred embodiment, the formulations contain about 50% w/w or about 55 w/w ethanol and 5 w/w propylene glycol as co-solvent.

Suitable antioxidants include butylated hydroxyanisole (BHA), butylated hydroxytoluene (BHT), methionine, sodium ascorbate, sodium thiosulfate, thioglycerol, ascorbic acid, propyl gallate, dL-alpha-tocopherol, sodium sulfite, sodium metabisulfite, sodium bisulfite cysteine hydrochloride, glutathione and a combination thereof. Presently preferred antioxidants include BHA, BHT, sodium thiosulfate, dL alpha-tocopherol (Vitamin E) and sodium ascorbate.

In a preferred embodiment, the amount of antioxidant included in the formulation is from about 0.001% to about 0.5% w/w.

In another preferred embodiment, the amount of antioxidant is about 0.01% w/w of BHA.

In an alternative embodiment, the antioxidant is a mixture of about 0.01% w/w of BHA and about 0.005% w/w of BHT.

In yet another embodiment, the antioxidant is about 0.01% w/w of sodium thiosulfate.

In a preferred embodiment, the antioxidant is about 0.3% w/w dL alpha-tocopherol.

In a most preferred embodiment, the antioxidant is about 0.02% w/w of sodium ascorbate.

In the present formulations, water is used as the solvent. Preferably, from about 10% to about 99% w/w water is in the formulations. More preferably, from about 20% to about 80% w/w water is in the formulations. Even more preferably, from about 25% to about 45% w/w water is in the formulations. In most preferred embodiments, the formulations contain about from about 30% to about 40% w/w water, or about 29.8%, 33.2%, 31.32%, 34.5% or 37.5% w/w water.

In a preferred embodiment, the formulations of the present invention have a pH of from about 2 to about 7. In a more preferred embodiment, the formulations of the present invention have a pH of from about 3 to about 6, even more preferably from about 4 to about 5.

In a most preferred embodiment, the formulations of the present invention have a pH of about 4.0±0.2 or 4.5±0.2.

In another preferred embodiment, the formulations contain ethanol as the co-solvent.

In yet another preferred embodiment, the formulations contain propylene glycol as the co-solvent.

In a more preferred embodiment, the formulations contain a mixture of ethanol and propylene glycol as the co-solvent.

In another embodiment, the formulations of the present invention contain a chelating agent. In a preferred embodiment, the chelating agent is edetate disodium dihydrate, (also known as edetate disodium or EDTA) preferably at a concentration from about 0.001% to about 0.5% w/w.

In an embodiment, the formulations contain from about 0.0001% to about 0.5% w/w of the chelating agent. In a preferred embodiment, the formulations contain from about 0.0005% w/w to about 0.10% of the chelating agent. In a more preferred embodiment, the formulations contain from about 0.001% to about 0.05 w/w of the chelating agent. In a more preferred embodiment, the formulations contain from about 0.001 to about 0.008% w/w of the chelating agent. In a most preferred embodiment, the formulations contain about 0.001% or 0.005% of the chelating agent.

In a preferred embodiment, the present invention is directed to sublingual spray formulation comprising naloxone, a pharmaceutically acceptable salt or a derivative thereof, in an amount from about 0.01% to about 20% w/w, water in an amount from about 10% to about 95% w/w, a cosolvent in an amount from about 5% to about 90% w/w, a permeation enhancer in an amount of 0.001% to 10% w/w, an antioxidant in an amount from about 0.0001% to about 0.1% w/w, and a chelating agent in an amount from about 0.0001% to 0.5% w/w, wherein the % w/w is of the total formulation.

In a preferred embodiment, the permeation enhancer is selected from the group consisting of menthol in an amount from about 0.001% to about 10.0% w/w caprylic acid in an amount from about 0.1% to 10% w/w, benzalkonium chloride (“BKC”) in an amount from about 0.001% to 10% w/w and a combination thereof. In another preferred embodiment, the formulation contains edetate disodium dihydrate as the chelating agent at 0.001% w/w or 0.005% w/w.

In a preferred embodiment, the present invention is directed to sublingual spray formulation comprising naloxone, a pharmaceutically acceptable salt or a derivative thereof, in an amount from about 0.01% to about 20% w/w, water in an amount from about 10% to about 95% w/w, a cosolvent in an amount from about 5% to about 90% w/w, a permeation enhancer in an amount from about 0.001% to about 10% w/w.

In yet another embodiment, the present invention is directed to naloxone, a pharmaceutically acceptable salt or a derivative thereof, in an amount from about 1% to about 15% w/w, water in an amount from about 20% to about 80% w/w, a cosolvent in an amount from about 5% to about 90% w/w, an antioxidant in an amount from about 0.001% to about 0.1% w/w, and a chelating agent in an amount from about 0.001% to 0.5%, wherein the % w/w is of the total formulation. In a preferred embodiment of the formulation, naloxone is a salt. In yet another preferred embodiment, the formulation further comprises a permeation enhancer selected from menthol in an amount from about 0.01% to about 10% w/w, caprylic acid in an amount from about 0.1% to 10% w/w, BKC in an amount from about 0.001% to 10% w/w, and a combination thereof. In another preferred embodiment, the chelating agent is edetate disodium dihydrate, preferably at a concentration from about 0.001% to about 0.5% w/w.

In yet another embodiment, the present invention is directed to naloxone, a pharmaceutically acceptable salt or a derivative thereof, in an amount from about 1% to about 20% w/w, water in an amount from about 20% to about 80% w/w, a cosolvent in an amount from about 5% to about 90% w/w, a permeation enhancer in an amount from about 0.001% to about 10% w/w. Preferably the formulation further comprises a chelating agent in an amount from about 0.001% to 0.5%, In a preferred embodiment of the formulation, naloxone is a salt. In another preferred embodiment, the permeation enhancer is selected from menthol, caprylic acid, BKC and a combination thereof. In a more preferred embodiment the formulation further comprises a chelating agent, preferably edetate disodium dihydrate (EDTA).

In a further embodiment, the present invention is directed to naloxone, a pharmaceutically acceptable salt or a derivative thereof in an amount from about 1% to about 12% w/w, water in an amount from about 25% to about 45% w/w, a cosolvent in an amount from about 40% to about 75% w/w, an antioxidant in an amount from about 0.005% to about 0.08% w/w, a permeation enhancer in an amount from about 0.001% to about 10% w/w, and a chelating agent in an amount from about 0.001% to 0.5% w/w, wherein the % w/w is of the total formulation. In a preferred embodiment of this formulation, naloxone is a salt. In another preferred embodiment, the formulation also contains menthol in an amount from about 0.02% to about 0.08% w/w. In yet another preferred embodiment, the formulation contains edetate disodium dihydrate as the chelating agent.

In a preferred embodiment, the formulations of the present invention are directed to sublingual spray formulations wherein the amount of naloxone salt is from about 2% to about 3% w/w, the amount of water is from about 35% to about 40% w/w, the amount of cosolvent is from about 50% to about 70% w/w, the amount of permeation enhancer is from 0.01% to about 2.5% w/w, and the amount of antioxidant is about 0.01% w/w, wherein the % w/w is of the total formulation. Preferably, the cosolvents are ethanol and propylene glycol and the ethanol is from about 50% to about 60% w/w and the propylene glycol is from about 3% to about 7% w/w. Preferably, the formulation contains about 8% w/w naloxone hydrochloride, about 37.5% w/w water, about 55% w/w ethanol, and about 5% propylene glycol. In another embodiment, the formulation also contains about 0.01% to 1% w/w menthol, about 0.1% to 10% w/w caprylic acid, about 0.001 to 0.2% w/w BKC or combination thereof as permeation enhancers and about 0.005% or 0.02% w/w disodium edetate dihydrate as chelating agent.

In an embodiment, the formulations of the present invention are directed to sublingual spray formulations wherein the amount of naloxone salt is from about 5% to about 10% w/w, the amount of water is from about 30% to about 35% w/w, the ethanol is from about 50% to about 60% w/w, the propylene glycol is from about 3% to about 7% w/w, and the antioxidant is from about 0.01% to about 0.03% w/w, wherein the % w/w is of the total formulation. Preferably, this formulation contains about 6.7% w/w naloxone salt, about 33.2% w/w water, about 55% w/w ethanol, and about 5% propylene glycol. In an embodiment, the formulation also contains about 0.01% to 1% w/w menthol, about 0.1% to 10% w/w caprylic acid, about 0.001 to 0.2% w/w BKC or combination thereof as permeation enhancers and about 0.005% w/w or 0.02% disodium edetate dihydrate.

In a preferred embodiment, the formulations of the present invention are directed to sublingual spray formulations wherein the amount of naloxone is from about 3% to about 5% w/w, the amount of water is from about 35% to about 40% w/w, the amount of cosolvent is from about 50% to about 70% w/w, and the amount of antioxidant, sodium ascorbate, is about 0.02% w/w, wherein the % w/w is of the total formulation. Preferably, the cosolvents in this formulation are ethanol and propylene glycol and the ethanol is from about 45% to about 55% w/w and the propylene glycol is from about 3% to about 7% w/w. Preferably, the formulation contains about 4% w/w naloxone, about 34.5% w/w water, about 50% w/w ethanol, and about 5% propylene glycol. In another embodiment, the formulation also contains about 0.01% to 1% w/w menthol, about 0.1% to 10% w/w caprylic acid, about 0.001 to 0.2% w/w BKC or a combination thereof as permeation enhancers and about 0.005% or 0.02% w/w disodium edetate dihydrate.

In yet another embodiment, the formulations of the present invention are directed to sublingual spray formulations wherein the amount of naloxone is from about 8% to about 12% w/w, the amount of water is from about 25% to about 35% w/w, the amount of ethanol is from about 50% to about 60% w/w, the amount of propylene glycol is from about 3% to about 7% w/w, and the amount of antioxidant is from about 0.01% to about 0.03% w/w, wherein the % w/w is of the total formulation. Preferably, the formulation contains about 10.1% w/w naloxone, about 29.8% w/w water, about 55% w/w ethanol, and about 5% propylene glycol. In an embodiment, the formulation also contains about 0.01% to 1% w/w menthol, about 0.1% to 10% w/w caprylic acid, about 0.001 to 0.2% w/w BKC or combination thereof as permeation enhancers and about 0.005% or 0.02% w/w disodium edetate dihydrate.

In most preferred embodiment, the formulations of the present invention are directed to sublingual spray formulations wherein the amount of naloxone is from about 8% to about 12% w/w, the amount of water is from about 25% to about 35% w/w, the amount of ethanol is from about 50% to about 60% w/w, the amount of propylene glycol is from about 3% to about 7% w/w, and the amount of antioxidant is from about 0.01% to about 0.03% w/w, wherein the % w/w is of the total formulation. Preferably, the formulation contains about 10.26% w/w naloxone, about 31.32% w/w water, about 50% w/w ethanol, 5% propylene glycol, about 0.001% w/w edetate disodium dihydrate, about 0.02% w/w sodium ascorbate, and 0.01% w/w benzalkonium chloride, about 0.8% w/w sucralose, about 2% w/w caprylic acid, about 0.5% w/w L-menthol, and about 0.08% flavoring agent.

In some embodiments, the formulations of the present invention contain citric acid or sodium hydroxide solution as a pH adjustor.

Pharmaceutically acceptable salts that can be used in accordance with the current invention include but are not limited to hydrochloride, hydrochloride dihydrate, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts.

In preferred embodiments the pharmaceutically acceptable salt is hydrochloride.

Derivatives of naloxone that can be used in accordance with the current invention include but are not limited 3-O-acyl, phenylhydrazone, and methiodide derivatives.

The solvent used with the present invention is United States Pharmacopeia (“USP”) purified water.

Cosolvents that can be used in accordance with the current invention are alcohols, and glycols or a mixture thereof

Alcohols that can be used in accordance with the current invention include but are not limited to methanol, ethanol (also known as dehydrated alcohol), propyl alcohol, and butyl alcohol.

Glycols that can be used in accordance with the current invention include but are not limited to propylene glycol, polypropylene glycol, and butylene glycol and polyethylene glycols such as PEG 200, PEG 300, PEG 400 and PEG 600 and the like.

In preferred embodiments, the cosolvent is ethanol or propylene glycol or a mixture thereof

In another preferred embodiment, the amount of cosolvent included in the formulation is from about 5% to about 90% w/w. In other more preferred embodiments, the amount of cosolvent included in the formulation is about 5% w/w propylene glycol. In other more preferred embodiments, the amount of cosolvent included in the formulation is about 50% w/w ethanol.

In other more preferred embodiments the cosolvent is a mixture of propylene glycol at about 5% w/w and ethanol at about 50% w/w.

Solubilizers that can be used in accordance with the current invention are hydroxpropyl beta-cyclodextrin (“HPβCD”) and sulfobutylether cyclodextrin or a mixture thereof

In preferred embodiments the solubilizer is HPβCD.

In more preferred embodiments the amount of HPβCD is about 30% w/w.

Permeation enhancers that can be used in accordance with the current invention include but are not limited to menthol, limonene, carvone, methyl chitosan, polysorbates including Tween® 80 (polysorbate 80; Tween is a registered trademark of Uniqema Americas, LLC), sodium lauryl sulfate, glyceryl oleate, caprylic acid, pelargonic acid, capric acid, undecylenic acid, lauric acid, myristic acid, palmitic acid, oleic acid, stearic acid, linolenic acid, arachidonic acid, benzalkonium chloride (BKC), cetylpyridium chloride, edetate disodium dihydrate, sodium desoxycholate, sodium deoxyglycolate, sodium glycocholate, sodium caprate, sodium taurocholate, sodium hydroxybenzoyal amino caprylate, dodecyl dimethyl aminopropionate, L-lysine, glycerol oleate, glyceryl monostearate, citric acid, and peppermint oil. Preferably the permeation enhancer is selected from the group consisting of menthol, benzalkonium chloride, edetate disodium dihydrate, caprylic acid, and a combination thereof.

In preferred embodiments, the amount of permeation enhancer is from about 0.001% to about 10% w/w. In a more preferred embodiment, the formulations contain from about 0.01% to about 5.0% w/w permeation enhancer. In a preferred embodiment, the formulations contain from about 0.02% to about 2.0% w/w permeation enhancer. In a most preferred embodiment, the formulations contain 2.0% w/w permeation enhancer.

In preferred embodiment the permeation enhancer is L-menthol, caprylic acid, BKC, edetate disodium dihydrate (EDTA) or combination thereof, the preferred amount of L-menthol is from about 0.001% to about 10.0% w/w, caprylic acid is from about 0.1% to about 10% w/w, BKC is from about 0.001 to about 10% w/w, EDTA is from about 0.0005% to 0.1% w/w. In a more preferred embodiment, the formulations contain from about 0.01% to about 0.5% w/w L-menthol, about 0.5% to about 5% w/w caprylic acid, about 0.005 to about 0.1% w/w BKC, about 0.0002% to about 0.01% w/w EDTA or a combination thereof. In an even more preferred embodiment, the formulations contain from about 0.02% to about 0.5% w/w L-menthol, about 1% to about 2% w/w caprylic acid, about 0.01 to about 0.1% w/w BKC, about 0.001 to about 0.005 w/w EDTA or a combination thereof. In a most preferred embodiment, the formulations contain about 0.5% w/w L-menthol, about 2% w/w caprylic acid, about 0.01% w/w BKC, about 0.001% edetate disodium dihydrate EDTA or a combination thereof.

In yet another embodiment, the permeation enhancer is about 0.5% w/w of menthol.

In yet another preferred embodiment, the permeation enhancer is about 2.0% w/w caprylic acid.

In a most preferred embodiment, the permeation enhancer is about 0.01% w/w of benzalkonium chloride (BKC).

In a most preferred embodiment, the permeation enhancer is about 0.001% w/w of edetate disodium dihydrate (EDTA).

Formulations of the present invention may have a pH range from about 2.0 to about 7.0, preferably from about 3 to about 6 and more preferably from about 4.0 to about 5.0 pH, most preferably 4.5±01, adjustors that can be used in accordance with the present invention include but are not limited to citric acid and sodium hydroxide. In preferred embodiments the amount of sodium hydroxide or citric acid is from about 0.002% to about 0.03% w/w. In more preferred embodiments the amount of sodium hydroxide is about 0.015% w/w. In other more preferred embodiments the amount of sodium hydroxide is about 0.012% w/w.

In a further embodiment, the formulation contains a permeation enhancer, a sweetener, a sweetness enhancer, a pH modifier, a flavoring agent, a preservative, or a combination thereof

In a preferred embodiment, the formulations contain a sweetener. In a more preferred embodiment, the sweetener is selected from the group consisting of sucralose, aspartame, saccharin, dextrose, mannitol, glycerin, and xylitol. In a preferred embodiment, the formulations contain from about 0.001% w/w to about 2% w/w of sweetener. In a more preferred embodiment, the formulations contain from about 0.05% w/w to about 1% w/w of the sweetener. In a most preferred embodiment, the formulations contain sucralose as sweetener at about 0.8% w/w.

In another embodiment, the formulations contain a flavoring agent. In a preferred embodiment, the formulations contain a flavoring agent selected from the group consisting of peppermint oil, menthol, spearmint oil, citrus oil, cinnamon oil, strawberry flavor, cherry flavor, raspberry flavor, orange oil, and a combination thereof. Other appropriate flavoring agents known by those of skill in art could also be added to formulations of the present invention. In a preferred embodiment, the formulations contain from about 0.001% w/w to about 1% w/w of the flavoring agent. In a more preferred embodiment, the formulations contain from about 0.005% w/w to about 0.5% w/w of the flavoring agent. In a most preferred embodiment, the formulations contain strawberry as flavoring agent at about 0.08% w/w.

In yet another embodiment, the formulations of the present invention are capable of producing a droplet size distribution wherein the mean Dv(10) is from about 12 to about 35 microns during administration.

In a further embodiment, the formulations of the present invention are capable of producing a droplet size distribution wherein the mean Dv(50) is from about 30 to about 55 microns during administration.

In yet another embodiment, the formulations of the present invention are capable of producing a droplet size distribution wherein the mean Dv(90) is from about 75 to about 600 microns during administration. Preferably, the formulations of the present invention are capable of producing a droplet size distribution wherein the mean Dv(90) is from about 85 to about 500 microns during administration.

Formulations without Alcohol

In a further embodiment, the invention is directed to storage stable sublingual spray formulations comprising an effective amount of naloxone, a pharmaceutically acceptable salt or a derivative thereof, water, and an antioxidant, and the formulations do not contain alcohol.

In a further embodiment, the invention is directed to storage stable sublingual spray formulations comprising an effective amount of naloxone, a pharmaceutically acceptable salt or a derivative thereof, water, and a permeation enhancer, and the formulations do not contain alcohol.

In a preferred embodiment, the sublingual spray formulation comprises from about 0.01% w/w to about 20% w/w naloxone or a salt or derivative thereof. In a more preferred embodiment, the sublingual spray formulation comprises from about 1% w/w to about 12% w/w naloxone or a salt or derivative thereof. In a most preferred embodiment, the formulations contain from about 3% w/w to about 6% w/w naloxone or a salt or derivative thereof

In another embodiment, the formulations contain from about 20% w/w to about 99% water. In a preferred embodiment, the formulations contain from about 30% w/w to about 98% w/w water. In a more preferred embodiment, the formulations contain from about 80% w/w to about 99% w/w water. In a most preferred embodiment, the formulations contain from about 88% w/w to about 99% w/w water.

In an embodiment, the formulations contain from about 5% w/w to about 50% w/w glycerol. In a preferred embodiment, the formulations contain from about 10% w/w to about 40% w/w glycerol. In a more preferred embodiment, the formulations contain form about 15% w/w to about 35% w/w glycerol.

In another embodiment, the formulations contain from about 0.1% w/w to about 50% w/w polyethylene glycol 400. In a more preferred embodiment, the formulations contain from about 10% w/w to about 40% w/w polyethylene glycol 400.

In another embodiment, the formulations contain from about 0.1% w/w to about 50% w/w propylene glycol. In a more preferred embodiment, the formulations contain from about 10% w/w to about 40% w/w propylene glycol.

In another embodiment, the formulation contains a pharmaceutically acceptable salt of naloxone. In a preferred embodiment, the formulation contains a salt selected from the group consisting of hydrochloride, citrate, halide, phosphate, sulfate, acetate, ascorbate, maleate, succinate, carbonate, mesylate and lactate. One of skill in the art could use other pharmaceutically acceptable naloxone salts in the formulations of the present invention.

In a preferred embodiment, the antioxidant is selected from the group consisting of ascorbic acid, cysteine HCl monohydrate, citric acid, ethylenediamine tetra acetic acid (EDTA), methionine, sodium citrate, sodium ascorbate, sodium thiosulfate, sodium metabisulfite, sodium bisulfite, glutathione and thioglycerol. Other appropriate antioxidants known by those of skill in the art could also be added to formulations of the present invention.

In a preferred embodiment, the formulations contain from about 0.0001% w/w to about 0.5% w/w of the antioxidant. In a more preferred embodiment, the formulations may contain from about 0.005% w/w to about 0.1% w/w of the antioxidant. In a most preferred embodiment, the formulations contain 0.05% of the antioxidant.

In another embodiment, the formulations of the present invention contain a chelating agent. In a preferred embodiment, the chelating agent is edetate disodium dihydrate

In an embodiment, the formulations contain from about 0.0001% to about 0.5% w/w of the chelating agent. In a preferred embodiment, the formulations contain from about 0.001% to about 0.50% w/w of the chelating agent. In a more preferred embodiment, the formulations contain about 0.05% w/w of the chelating agent.

In a further embodiment, the formulation contains a permeation enhancer, a sweetener, a sweetness enhancer, a pH modifier, a flavoring agent, a preservative, or a combination thereof

In another embodiment, the formulation contains a permeation enhancer. In a preferred embodiment, the permeation enhancer is selected from the group consisting of menthol, limonene, carvone, methyl chitosan, caprylic acid pelargonic acid, capric acid, undecylenic acid, lauric acid, myristic acid, palmitic acid, oleic acid, stearic acid, linolenic acid, arachidonic acid, polysorbates including Tween® 80, sodium edetate, benzalkonium chloride (BKC), cetylpyridinium chloride, sodium lauryl sulfate, citric acid, sodium desoxycholate, sodium deoxyglycolate, glyceryl oleate, glyceryl monosterate, Sodium hydroxybenzoyal amino caprylate, sodium caprate, dodecyl dimethyl aminopropionate, L-lysine, sodium glycocholate, citric acid, peppermint oil and a combination thereof. In a more preferred embodiment, the permeation enhancer is selected from the group consisting of polysorbates including Tween® 80, sodium edetate, benzalkonium chloride (BKC), cetylpyridinium chloride, sodium lauryl sulfate, citric acid, sodium desoxycholate, sodium deoxyglycolate, glyceryl oleate, glyceryl monosterate, L-lysine, sodium glycocholate, sodium taurocholate, citric acid, and a combination thereof. In an even more preferred embodiment, the permeation enhancer is selected from the group consisting of menthol, caprylic acid and BKC.

In preferred embodiments, the amount of permeation enhancer is from about 0.001% to about 10% w/w. In a more preferred embodiment, the formulations contain from about 0.001% to about 2.5% w/w permeation enhancer. In a most preferred embodiment, the formulations contain from about 0.02% to about 2.0% w/w permeation enhancer.

In preferred embodiment the permeation enhancer is menthol, caprylic acid, BKC or combination thereof, the preferred amount of L-menthol is from about 0.001% to about 10% w/w, caprylic acid is from about 0.1% to 10% w/w, BKC is from about 0.001 to 10% w/w. In a more preferred embodiment, the formulations contain from about 0.01% to about 0.5% w/w L-menthol, about 0.5% to 5% w/w caprylic acid, about 0.005 to 0.1% w/w BKC. In an even more preferred embodiment, the formulations contain from about 0.02% to about 0.5% w/w L-menthol, about 1% to 2% w/w caprylic acid, about 0.01 to 0.1% w/w BKC. In a most preferred embodiment, the formulations contain about 0.5% w/w L-menthol, about 2% w/w caprylic acid and about 0.005 w/w BKC.

In yet another embodiment, the permeation enhancer is about 0.5% w/w of menthol.

In yet another preferred embodiment, the permeation enhancer is about 2.0% w/w caprylic acid.

In a most preferred embodiment, the permeation enhancer is about 0.01% w/w of benzalkonium chloride (BKC).

In a preferred embodiment, the formulations contain a sweetener. In a more preferred embodiment, the sweetener is selected from the group consisting of sucralose, aspartame, saccharin, dextrose, mannitol, glycerin, and xylitol. In a preferred embodiment, the formulations contain from about 0.001% w/w to about 2% w/w of sweetener. In a more preferred embodiment, the formulations contain from about 0.05% w/w to about 1% w/w of the sweetener. In a most preferred embodiment, the formulations contain sucralose as sweetener at about 0.8% w/w.

In a further embodiment, the formulation may contain a sweetness enhancer, an ammonium salt form of crude and refined Glycyrrhizic Acid, for example, Magnasweet® product (available from Mafco Worldwide Corporation, Magnasweet is a registered trademark of Mafco Worldwide Corporation). Magnasweet® products use the ammonium salt forms of crude and refined Glycyrrhizic Acid. Glycyrrhizic Acid is also available as a pure derivative in the sodium and potassium salt forms.

In another embodiment, the formulations contain a pH modifier. In a preferred embodiment, the pH modifier adjusts the pH of the formulation to from about 2 to about 7. In a more preferred embodiment, the pH modifier adjusts the pH of the formulation to from about 3 to about 6, from about 4 to about 5 or from about 2 to about 4. In most preferred embodiments, the pH modifier adjusts the pH of the formulations to about 2.5 or 3 or 4.5±0.1.

In another embodiment, the formulations contain a flavoring agent. In a preferred embodiment, the formulations contain a flavoring agent selected from the group consisting of peppermint oil, menthol, spearmint oil, citrus oil, cinnamon oil, strawberry flavor, cherry flavor, raspberry flavor, orange oil, and a combination thereof. Other appropriate flavoring agents known by those of skill in art could also be added to formulations of the present invention. In a preferred embodiment, the formulations contain from about 0.001% w/w to about 1% w/w of the flavoring agent. In a more preferred embodiment, the formulations contain from about 0.005% w/w to about 0.5% w/w of the flavoring agent. In a most preferred embodiment, the formulations contain strawberry as flavoring agent at about 0.08% w/w.

In yet another embodiment, the formulations may contain a preservative. In a preferred embodiment, the preservative is selected from the group consisting of butyl paraben, methyl paraben, ethyl paraben, propyl paraben, sodium benzoate, and benzoic acid. In a preferred embodiment, the formulations contain from about 0.001% w/w to about 1% w/w of the preservative. In a more preferred embodiment, the formulations contain from about 0.005% w/w to about 0.2% w/w of the preservative. In a most preferred embodiment, the formulations contain methyl paraben as a preservative at about 0.1% w/w.

In yet another embodiment, the formulations of the present invention are capable of producing a droplet size distribution wherein the mean Dv(10) is from about 12 to about 20 microns during administration.

In a further embodiment, the formulations of the present invention are capable of producing a droplet size distribution wherein the mean Dv(50) is from about 25 to about 35 microns during administration.

In yet another embodiment, the formulations of the present invention are capable of producing a droplet size distribution wherein the mean Dv(90) is from about 40 to about 150 microns during administration. Preferably, the formulations of the present invention are capable of producing a droplet size distribution wherein the mean Dv(90) is from about 60 to about 110 microns during administration.

All claims, aspects and embodiments of the invention, and specific examples thereof, are intended to encompass equivalents thereof.

In a further embodiment, the invention is directed to treating patients by administering the formulations (with or without alcohol) of the present invention to the patient. In preferred embodiment, the formulations are administered in order to treat opioid dependence, opioid overdose, and congenital insensitivity to pain with anhidrosis.

### DEFINITIONS

As used herein, all numerical values relating to amounts, weights, and the like, that are defined as “about” each particular value is plus or minus 10%. For example, the phrase “about 10% w/w” is to be understood as “9% to 11% w/w.” Therefore, amounts within 10% of the claimed value are encompassed by the scope of the claims.

As used herein “% w/w” refers to the percent weight of the total formulation.

As used herein the term “effective amount” refers to the amount necessary to treat a patient in need thereof.

As used herein the term “patient” refers but is not limited to a person that is being treated opioid dependence, opioid overdose, insensitivity to pain with anhidrosis, or another affliction or disease that can be treated with naloxone.

As used herein the phrase “pharmaceutically acceptable” refers to ingredients that are not biologically or otherwise undesirable in a sublingual dosage form.

As used herein, “storage stable” refers to formulations which maintain greater than 95% purity following at least four weeks of storage at about 40° C.

Preferably, the (alcohol and alcohol-free) formulations of the present invention are propellant free. As used herein, “propellant free” refers to a formulation that is not administered using compressed gas.

The following examples are intended to illustrate the present invention and to teach one of ordinary skill in the art how to make and use the invention. They are not intended to be limiting in any way.

### EXAMPLES

### Example 1

### Preparation of Naloxone Formulations Containing Alcohol

Sublingual spray formulations were created by first degassing ethanol and USP purified water, separately. Next, the ethanol and purified water were each purged with nitrogen. Soluble excipients were then dissolved in either the ethanol or the purified water based on their solubility. Next, the solutions were combined. Naloxone was added to the final solution and mixed until dissolved.

Strawberry flavor was used as the source of the flavoring agent.

TABLE 1
Stable Sublingual Naloxone Spray Formulations

| Formulation | Control | #1A | #2A | #3A | #4A | #5A | #6A | #7A |
|---|---|---|---|---|---|---|---|---|
| Naloxone | 2.44 | 2.44 | 2.44 | 2.44 | 2.44 | 4.00 | 6.7 | 10.1 |
| Water (USP) | 37.56 | 37.55 | 37.55 | 37.54 | 37.54 | 34.45 | 33.23 | 29.83 |
| Alcohol | 55 | 55 | 55 | 55 | 55 | 55 | 55 | 55 |
| Propylene Glycol | 5 | 5 | 5 | 5 | 5 | 5 | 5 | 5 |
| L-menthol | | | | | | 0.05 | | |
| Sodium Thiosulfate | | 0.01 | 0.01 | | | | | |
| Citric Acid | | 0.0025 | | | | | | |
| Flavoring agent | | | | | | 0.08 | | |
| Edetate disodium | | | 0.005 | 0.005 | 0.005 | 0.005 | 0.005 | 0.005 |
| dihydrate | | | | | | | | |
| BHA | | | | 0.01 | | | | |
| BHT | | | | 0.005 | | | | |
| Sodium Ascorbate | | | | | 0.02 | 0.02 | 0.02 | 0.02 |

values = % w/w

### Example 2

### Stability Testing of Naloxone Formulations

The formulations listed in Table 1 were subjected to stability testing at 40° C. and 55° C.±2° C. under 75%±5% relative humidity for eight weeks. The stability data was collected at zero, one, two, three, four, and eight weeks at 55° C. and at zero, four, and eight weeks at 40° C. Assay and impurities were detected using high performance liquid chromatography with an ultraviolet detector. The assay was performed at 288 nm and indicated as a % of initial concentration. For all impurities, analysis was performed at 240 nm and expressed as a % area. Amounts of particular impurities are listed in Tables 2A to 2F and 3A to 3H as a percentage of the area of each formulation along with amount of total impurities. “BQL” refers to “Below Quantifiable Limit” and “ND” refers to “Not Detected.”

### Tables 2A to 2E. Stability Data for Sublingual Naloxone Spray Formulations Stored at 40° C.±2° C. Under 75%±5% Relative Humidity

### 2A. Stability of Control Stored at 40° C.

| Naloxone | RRT | T = 0 | 4 Weeks | 8 Weeks |
|---|---|---|---|---|
| Assay (%) | | | | |
| Impurity C | 0.66 | ND | 0.81% | 0.92% |
| Impurity A | 0.83 | ND | 0.37% | 0.51% |
| Impurity F | 0.93 | ND | ND | ND |
| Impurity D | 1.14 | ND | ND | ND |
| Impurity E | 2.85 | ND | 5.59% | 5.53% |
| Impurity B | 3.21 | ND | ND | ND |
| Unknown Impurities | 0.30 | ND | 0.13% | 0.18% |
| | 0.35 | ND | ND | ND |
| | 0.50 | ND | 0.28% | 0.46% |
| | 2.85 | ND | ND | ND |
| Total Impurities | | 0.00% | 7.18% | 7.60% |

### 2B. Stability of Form. #1A (with Sod. Thiosulphate & Citric Acid) Stored at 40° C.

| Naloxone | RRT | T = 0 | 4 Weeks | 8 Weeks |
|---|---|---|---|---|
| Assay (%) | | | | |
| Impurity C | 0.66 | ND | BQL | BQL |
| Impurity A | 0.83 | ND | BQL | BQL |
| Impurity F | 0.93 | ND | ND | ND |
| Impurity D | 1.14 | ND | ND | ND |
| Impurity E | 2.85 | ND | ND | ND |
| Impurity B | 3.21 | ND | ND | ND |
| Unknown Impurities | 0.32 | ND | ND | ND |
| | 0.52 | ND | ND | ND |
| | 2.67 | ND | ND | ND |
| | | ND | ND | ND |
| Total Impurities | | 0.00% | 0.00% | 0.00% |

### 2C. Stability of Form. #2A (with Sod. Thiosulphate & Edetate Disodium Dihydrate) Stored at 40° C.

| Naloxone | RRT | T = 0 | 4 Weeks | 8 Weeks |
|---|---|---|---|---|
| Assay (%) | | | | |
| Impurity C | 0.66 | ND | BQL | BQL |
| Impurity A | 0.83 | ND | BQL | BQL |
| Impurity F | 0.93 | ND | ND | ND |
| Impurity D | 1.14 | ND | ND | ND |
| Impurity E | 2.85 | ND | ND | ND |
| Impurity B | 3.21 | ND | ND | ND |
| | 0.50 | ND | ND | ND |
| Unknown Impurities | 0.52 | ND | ND | ND |
| | 0.74 | ND | ND | ND |
| | 1.37 | ND | ND | ND |
| | 2.20 | ND | ND | ND |
| | 2.47 | ND | ND | ND |
| Total Impurities | | 0.00% | 0.00% | 0.00% |

### 2D. Stability of Form. #3A (with BHA & BHT) Stored at 40° C.

| Naloxone | RRT | T = 0 | 4 Weeks | 8 Weeks |
|---|---|---|---|---|
| Assay (%) | | | | |
| Impurity C | 0.66 | ND | BQL | BQL |
| Impurity A | 0.83 | ND | BQL | BQL |
| Impurity F | 0.93 | ND | ND | ND |
| Impurity D | 1.14 | ND | ND | ND |
| Impurity E | 2.85 | ND | ND | ND |
| Impurity B | 3.21 | ND | ND | ND |
| Unknown Impurities | 0.31 | ND | ND | ND |
| | 0.50 | ND | ND | ND |
| | 1.79 | ND | ND | ND |
| | 1.83 | ND | ND | ND |
| | 2.10 | ND | ND | ND |
| | | ND | ND | ND |
| Total Impurities | | 0.00% | 0.00% | 0.00% |

### 2E. Stability of Form. #4A (with Sod. Ascorbate and Edetate Disodium Dihydrate) Stored at 40° C.

| Naloxone | RRT | T = 0 | 4 Weeks | 8 Weeks |
|---|---|---|---|---|
| Assay (%) | | 100 | | |
| Impurity C | 0.66 | ND | BQL | BQL |
| Impurity A | 0.83 | ND | 0.15% | 0.19% |
| Impurity F | 0.93 | ND | ND | ND |
| Impurity D | 1.14 | ND | ND | ND |
| Impurity E | 2.85 | ND | ND | ND |
| Impurity B | 3.21 | ND | ND | ND |
| Unknown Impurities | 0.52 | ND | ND | BQL |
| | 1.79 | ND | ND | ND |
| | 1.83 | ND | ND | ND |
| | 2.10 | ND | ND | ND |
| Total Impurities | | 0.00% | 0.15% | 0.19% |

| Naloxone | RRT | T = 0 | 4 Weeks | 3 Months |
|---|---|---|---|---|
| Assay (%) | | 100 | 97.77 | 97.6 |
| Impurity C | 0.66 | ND | ND | 0.03 |
| Impurity A | 0.83 | 0.11 | 0.12 | 0.15 |
| Impurity F | 0.93 | ND | ND | ND |
| Impurity D | 1.14 | ND | ND | ND |
| Impurity E | 2.85 | ND | 0.13 | 0.13 |
| Impurity B | 3.21 | ND | ND | ND |
| Unknown | 0.52 | ND | ND | ND |
| Impurities | 0.79 | ND | ND | ND |
| | 1.83 | ND | ND | ND |
| | 2.10 | ND | ND | ND |
| | 2.32 | ND | ND | ND |
| | 3.23 | ND | ND | ND |
| | 3.94 | ND | ND | 0.04 |
| | 5.14 | ND | ND | ND |
| Total Impurities | | 0.11% | 0.25% | 0.29% |

Sublingual naloxone formulations of the present invention contained less than one percent total impurities after eight weeks at 40° C. This is a stark contrast to the control formulation which contained 7.6% impurities at the same time. Specifically, the formulations which contained sodium thiosulfate or BHA and BHT resulted in 0% detected impurities after eight weeks. Also, formulations which contain sodium ascorbate (0.02% wt/wt) and edetate disodium dihydrate (0.005% wt/wt) resulted in only 0.29% total impurities after 3 months.

### Tables 3A to 3H. Stability Data for Sublingual Naloxone Spray Formulations Stored at 55° C.±2° C.

### 3A. Stability of Control Stored at 55° C.

| Naloxone | RRT | T = 0 | 1 Week | 2 Weeks | 3 Weeks | 4 Weeks | 8 Weeks |
|---|---|---|---|---|---|---|---|
| Assay (%) | | 100.00 | | | | | |
| Impurity C | 0.66 | ND | ND | ND | 0.54% | 0.33% | 0.35% |
| Impurity A | 0.83 | ND | ND | ND | 1.31% | 1.39% | 1.59% |
| Impurity F | 0.93 | ND | ND | ND | ND | ND | ND |
| Impurity D | 1.14 | ND | ND | ND | ND | ND | ND |
| Impurity E | 2.85 | ND | ND | ND | ND | ND | ND |
| Impurity B | 3.21 | ND | ND | ND | ND | ND | ND |
| Unknown | 0.30 | ND | ND | ND | ND | 0.1 | 0.32% |
| Impurities | 0.35 | ND | ND | ND | 0.15% | 0.16% | 0.08% |
| | 0.50 | ND | ND | ND | 0.83% | 0.81% | 0.67% |
| | 2.85 | ND | ND | ND | 4% | 7.50% | 6.65% |

| | | | | | | |
|---|---|---|---|---|---|---|
| Total Impurities | 0.00% | 0.00% | 0.00% | 6.83% | 10.29% | 9.66% |

### 3B. Stability of Form. #1A (with Sod. Thiosulphate & Citric Acid) Stored at 55° C.

| Naloxone | RRT | T = 0 | 1 Week | 2 Weeks | 3 Weeks | 4 Weeks | 8 Weeks |
|---|---|---|---|---|---|---|---|
| Assay (%) | | 100.00 | | | | | |
| Impurity C | 0.66 | ND | ND | ND | 0.12% | 0.37% | 0.29% |
| Impurity A | 0.83 | ND | ND | ND | 0.14% | 0.67% | 1.01% |
| Impurity F | 0.93 | ND | ND | ND | ND | ND | ND |
| Impurity D | 1.14 | ND | ND | ND | ND | ND | ND |
| Impurity E | 2.85 | ND | ND | ND | 0.55% | 1.88% | 1.52% |
| Impurity B | 3.21 | ND | ND | ND | ND | ND | ND |
| Unknown | 0.32 | ND | ND | ND | ND | 0.09% | 0.25% |
| Impurities | 0.52 | ND | ND | ND | 0.06% | 0.51% | 0.59% |
| | 2.67 | ND | ND | ND | ND | ND | ND |
| | | ND | ND | ND | ND | ND | ND |

| | | | | | | |
|---|---|---|---|---|---|---|
| Total Impurities | 0.00% | 0.00% | 0.00% | 0.87% | 3.52% | 3.66% |

### 3C. Stability of Form. #2A (with Sod. Thiosulphate & Edetate Disodium Dihydrate) Stored at 55° C.

| Naloxone | RRT | T = 0 | 1 Week | 2 Weeks | 3 Weeks | 4 Weeks | 8 Weeks |
|---|---|---|---|---|---|---|---|
| Assay (%) | | 100.00 | | | | | |
| Impurity C | 0.66 | ND | ND | ND | ND | BQL | BQL |
| Impurity A | 0.83 | ND | ND | ND | BQL | 0.07% | 0.11% |
| Impurity F | 0.93 | ND | ND | ND | ND | ND | ND |
| Impurity D | 1.14 | ND | ND | ND | ND | ND | ND |
| Impurity E | 2.85 | ND | ND | ND | ND | ND | ND |
| Impurity B | 3.21 | ND | ND | ND | ND | ND | ND |
| | 0.31 | ND | ND | ND | ND | ND | ND |
| Unknown | 0.52 | ND | ND | ND | ND | ND | 0.08% |
| Impurities | 0.74 | ND | ND | ND | ND | ND | ND |
| | 1.37 | ND | ND | ND | ND | ND | ND |
| | 2.20 | ND | ND | ND | ND | ND | ND |
| | 2.47 | ND | ND | ND | ND | ND | ND |

| | | | | | | |
|---|---|---|---|---|---|---|
| Total Impurities | 0.00% | 0.00% | 0.00% | 0.00% | 0.07% | 0.19% |

### 3D. Stability of Form. #3A (with BHA & BHT) Stored at 55° C.

| Naloxone | RRT | T = 0 | 1 Week | 2 Weeks | 3 Weeks | 4 Weeks | 8 Weeks |
|---|---|---|---|---|---|---|---|
| Assay (%) | | 100.00 | | | | | |
| Impurity C | 0.66 | ND | ND | ND | ND | ND | BQL |
| Impurity A | 0.83 | ND | ND | ND | BQL | 0.07% | 0.13% |
| Impurity F | 0.93 | ND | ND | ND | ND | ND | ND |
| Impurity D | 1.14 | ND | ND | ND | ND | ND | ND |
| Impurity E | 2.85 | ND | ND | ND | ND | ND | ND |
| Impurity B | 3.21 | ND | ND | ND | ND | ND | ND |
| Unknown | 0.31 | ND | ND | ND | ND | ND | ND |
| Impurities | 0.50 | ND | ND | ND | ND | ND | 0.08% |
| | 1.79 | ND | ND | ND | ND | ND | ND |
| | 1.83 | ND | ND | ND | ND | ND | ND |
| | 2.10 | ND | ND | ND | ND | ND | ND |
| | | ND | ND | ND | ND | ND | ND |

| | | | | | | |
|---|---|---|---|---|---|---|
| Total impurities | 0.00% | 0.00% | 0.00% | 0.00% | 0.07% | 0.21% |

### 3E. Stability of Form. #4A (with Sod. Ascorbate and Edetate Disodium Dihydrate) Stored at

| Naloxone | RRT | T = 0 | 1 Week | 2 Weeks | 3 Weeks | 4 Weeks | 8 Weeks |
|---|---|---|---|---|---|---|---|
| Assay (%) | | 100.00 | | | | | |
| Impurity C | 0.66 | ND | ND | ND | ND | ND | 0.06% |
| Impurity A | 0.83 | ND | ND | ND | 0.11% | 0.19% | 0.19% |
| Impurity F | 0.93 | ND | ND | ND | ND | ND | ND |
| Impurity D | 1.14 | ND | ND | ND | ND | ND | ND |
| Impurity E | 2.85 | ND | ND | ND | ND | ND | ND |
| Impurity B | 3.21 | ND | ND | ND | ND | ND | ND |
| Unknown | 0.52 | ND | ND | ND | ND | ND | BQL |
| Impurities | 1.79 | ND | ND | ND | ND | ND | ND |
| | 1.83 | ND | ND | ND | ND | ND | ND |
| | 2.10 | ND | ND | ND | ND | ND | ND |

| | | | | | | |
|---|---|---|---|---|---|---|
| Total Impurities | 0.00% | 0.00% | 0.00% | 0.11% | 0.19% | 0.25% |

### 3F. Stability of Form. #5A (with Sod. Ascorbate and Edetate Disodium Dihydrate) Stored at

| | | | | 4 | | 8 |
|---|---|---|---|---|---|---|
| Naloxone | RRT | T = 0 | 2 Weeks | Weeks | 6 Weeks | Weeks |
| Assay (%) | | 100 | 102.37 | 98.75 | 98.51 | 100.76 |
| Impurity C | 0.66 | ND | ND | ND | ND | 0.05% |
| Impurity A | 0.83 | 0.11 | 0.14 | 0.15 | 0.19 | 0.17% |
| Impurity F | 0.93 | ND | ND | ND | ND | ND |
| Impurity D | 1.14 | ND | ND | ND | ND | ND |
| Impurity E | 2.85 | ND | 0.13 | 0.11 | 0.12 | 0.12% |
| Impurity B | 3.21 | ND | ND | ND | ND | ND |
| Unknown | 0.49 | ND | ND | ND | 0.06 | 0.05% |
| Impurities | 0.79 | ND | ND | ND | 0.03 | BQL |
| | 1.83 | ND | ND | ND | ND | ND |
| | 2.10 | ND | ND | ND | ND | ND |
| | 2.32 | ND | ND | ND | ND | ND |
| | 3.05 | ND | ND | ND | ND | ND |
| | 3.90 | ND | BQL | 0.05 | 0.07 | 0.05% |
| | 5.14 | ND | ND | ND | ND | ND |
| Total | | 0.11% | 0.27% | 0.31% | 0.47% | 0.44% |
| Impurities | | | | | | |

### 3H. Stability of Form. #6A (with Sod. Ascorbate and Edetate Disodium Dihydrate) Stored at

| Naloxone | RRT | T = 0 | 2 Weeks | 4 Weeks | 8 Weeks |
|---|---|---|---|---|---|
| Assay (%) | | 100.00 | 101.35 | 102.69 | 102.99 |
| Impurity C | 0.66 | ND | BQL | BQL | 0.08% |
| Impurity A | 0.81 | BQL | 0.08% | 0.19% | 0.18% |
| Impurity F | 0.93 | ND | ND | ND | ND |
| Impurity D | 1.14 | ND | ND | ND | ND |
| Impurity E | 2.85 | 0.04% | 0.07% | 0.06% | 0.10% |
| Impurity B | 3.21 | ND | ND | ND | 0.12% |
| Unknown | 0.33 | ND | ND | ND | ND |
| Impurities | 0.50 | ND | ND | ND | 0.06% |
| | 0.57 | ND | ND | ND | ND |
| | 0.61 | ND | ND | ND | ND |
| | 3.43 | ND | ND | ND | ND |
| Total Impurities | | 0.04% | 0.15% | 0.25% | 0.54% |

### 3G. Stability of Form. #7A (with Sod. Ascorbate and Edetate Disodium Dihydrate) Stored at

| | RRT | T = 0 | 2 Weeks | 4 Weeks | 8 Weeks |
|---|---|---|---|---|---|
| Assay (%) | | 100.00 | 100.91 | 100.92 | 102.05 |
| Impurity C | 0.66 | ND | 0.06% | 0.05% | 0.11% |
| Impurity A | 0.81 | BQL | 0.11% | 0.22% | 0.17% |
| Impurity F | 0.93 | ND | ND | ND | ND |
| Impurity D | 1.14 | ND | ND | ND | ND |
| Impurity E | 2.85 | 0.06% | 0.07% | 0.06% | 0.11% |
| Impurity B | 3.21 | ND | ND | ND | 0.13% |
| Unknown | 0.33 | ND | ND | ND | ND |
| Impurities | 0.50 | ND | ND | ND | 0.05% |
| | 0.57 | ND | ND | ND | ND |
| | 0.61 | ND | ND | ND | ND |
| | 2.41 | 0.00 | 0.00 | 0.00 | 0.05% |
| | 3.43 | ND | ND | ND | ND |
| Total Impurities | | 0.06% | 0.24% | 0.33% | 0.62% |

Similar to the stability study at 40° C., all of the formulations of the present invention had significantly fewer impurities at eight weeks compared to the control. The superior stability characteristics of the formulations of the present invention will allow the formulations to be effective when used by patients.

### Example 3

### Droplet Testing

In order to determine the spray profile of Formulation #5A, it was subjected to standardized droplet testing. A challenge of creating a Naloxone sublingual spray formulation is that it must be capable of producing spray droplets that are over 10 microns in diameter. Spray droplets 10 microns or smaller could be inhaled into the lungs. The optimal particle size for sublingual spray droplets is from 20 to about 200 microns in diameter. It is desirable for the formulation to have droplet sizes near 20 because this increases the surface area and increased surface area exposure is one factor that contributes to a high bioavailability. Sublingual formulations should be able to maintain a consistent droplet size throughout its shelf life.

Droplet analysis was conducted using standard laser analysis procedures known by those of skill in the art. Droplet size distribution (Dv10, Dv50, Dv90, and Span were tested at two distances, 3 cm and 6 cm). Dv10 refers to droplet size for which 10% of the total volume is obtained; Dv50 refers to droplet size for which 50% of the total volume is obtained; Dv90 refers to droplet size for which 90% of the total volume is obtained; Span refers to distribution span (Dv90-Dv10)/Dv50; % RSD refers to the percent relative standard deviation. The results of these tests can be seen below in Tables 4 to 9. Applicant found during testing that formulations of the present invention yielded desirable droplet sizes for sublingual administration. The testing also revealed that the formulation dose remains consistent when administered with a spray pump.

TABLE 4
Spray Profile of Naloxone Spray Formulation #5A,
Particle Size at 3 cm

| | | Particle Size | | | | |
|---|---|---|---|---|---|---|
| Formulation #5A | | DV(10) | DV(50) | DV(90) | % <10μ | Span |
| 3 cm | Actuation 1 | 14.79 | 28.92 | 389.9 | 1.225 | 12.97 |
| | Actuation 2 | 17.98 | 32.05 | 455.6 | 0.001 | 13.65 |
| | Actuation 3 | 13.46 | 36.92 | 584.8 | 4.747 | 15.48 |
| | Average | 15.41 | 32.63 | 476.8 | 1.991 | 14.03 |

TABLE 5
Spray Profile of Naloxone Spray Formulation #5A,
Particle Size at 6 cm

| | | Particle Size | | | | |
|---|---|---|---|---|---|---|
| Formulation #5A | | DV(10) | DV(50) | DV(90) | % <10μ | Span |
| 6 cm | Actuation 1 | 20.58 | 38.64 | 498.6 | 1.918 | 12.37 |
| | Actuation 2 | 18.67 | 37.59 | 529.4 | 1.537 | 13.59 |
| | Actuation 3 | 21.26 | 36.44 | 452.3 | 1.767 | 11.83 |
| | Average | 20.17 | 37.56 | 493.4 | 1.741 | 12.60 |

TABLE 6
Spray Profile of Naloxone Spray Formulation #5A,
Spray Pattern at 3 cm

| | | Spray Pattern | | |
|---|---|---|---|---|
| Formulation #5A | | Dmin (mm) | Dmax (mm) | Ovality Ratio |
| 3 cm | Actuation 1 | 21.2 | 33.4 | 1.577 |
| | Actuation 2 | 23.5 | 31.5 | 1.342 |
| | Actuation 3 | 17.6 | 30.9 | 1.755 |
| | Average | 20.8 | 31.9 | 1.558 |

TABLE 7
Spray Profile of Naloxone Spray Formulation #5A,
Spray Pattern at 6 cm

| | | Spray Pattern | | |
|---|---|---|---|---|
| Formulation #5A | | Dmin (mm) | Dmax (mm) | Ovality Ratio |
| 6 cm | Actuation 1 | 24.5 | 55.6 | 2.268 |
| | Actuation 2 | 34.3 | 49.7 | 1.447 |
| | Actuation 3 | 33.9 | 52 | 1.535 |
| | Average | 30.9 | 52.4 | 1.750 |

TABLE 8
Spray Profile of Naloxone Spray Formulation #5A,
Plume geometry data at 3 cm

Plume Geometry

| | | | Angle |
|---|---|---|---|
| Formulation #5A | | Width (mm) | (° ) |
| 3 cm | Actuation 1 | 28.7 | 51.1 |
| | Actuation 2 | 25.5 | 45.9 |
| | Actuation 3 | 35.4 | 60.4 |
| | Average | 29.9 | 52.5 |

TABLE 9
Spray Profile of Naloxone Spray Formulation #5A,
Plume geometry data at 6 cm

Plume Geometry

| | | |
|---|---|---|
| | | Angle |
| Formulation #5A | Width (mm) | (° ) |

| | | | |
|---|---|---|---|
| 6 cm | Actuation 1 | 54.3 | 48.4 |
| | Actuation 2 | 52.6 | 47.3 |
| | Actuation 3 | — | — |
| | Average | 53.5 | 47.9 |

As can be seen in Tables 4 to 9, Formulation #5A of the present invention provided excellent plume geometry and spray patterns.

### Example 4

### Preparation of Naloxone Formulations that are Alcohol-Free

In order to prepare a naloxone sublingual formulation, the components as indicated in “Table 10. The Components of Formulation #1AF” below were weighed. The components were mixed until a clear solution was formed.

Naloxone HCL dihydrate base U.S.P. was used as the source of naloxone in the formulations that follow. Methyl paraben, U.S.P., (available from Spectrum) was used as the preservative source. Strawberry flavor, Nat&Art 915.0543 U, (available from FONA) was used as the source of flavoring agent. Edetate Disodium Dihydrate, U.S.P., (available from Spectrum) was used as the source of chelating agent or as antioxidant. Water, U.S.P., purified, (available from RICCA) was used as the source of solvent.

TABLE 10
The Components of Formulation #1AF

| Ingredients | % w/w |
|---|---|
| Naloxone HCl Dihydrate | 4.82 |
| Sucralose | 0.80 |
| Methyl Paraben | 0.10 |
| Flavoring agent | 0.08 |
| Edetate Disodium Dihydrate | 0.05 |
| Water USP | 94.15 |
| | 100.0 |

### Example 5

### Preparation of Additional Naloxone Sublingual Formulations

In order to prepare naloxone sublingual formulations, the components as indicated in “Table 11. The Components of Control and Formulations #1AF to #6AF” below were weighed. The components were mixed until a clear solution was formed.

Strawberry flavoring was used as the source of flavoring agent.

TABLE 11
The Components of Control and Formulations #1AF to #6AF

| | Formulation | | | | | | |
|---|---|---|---|---|---|---|---|
| | Control | #1AF | #2AF | #3AF | #4AF | #5AF | #6AF |
| Naloxone HCL Dihydrate | 4.83 | 4.82 | 4.89 | 4.89 | 4.89 | 4.83 | 4.82 |
| Water (USP) | 94.19 | 94.15 | 95.01 | 94.08 | 93.98 | 94.16 | 94.15 |
| Sucralose | 0.8 | 0.8 | | 0.8 | 0.8 | 0.8 | 0.8 |
| Methyl Paraben | 0.1 | 0.1 | | 0.1 | 0.1 | 0.1 | 0.1 |
| Flavoring | 0.08 | 0.08 | 0.08 | 0.08 | 0.08 | 0.08 | 0.08 |
| Edetate Disodium Dihydrate | | 0.05 | 0.005 | 0.05 | 0.05 | 0.005 | 0.05 |
| L-cysteine Hydrochloride | | | | | 0.1 | | |
| Monohydrate | | | | | | | |
| Sodium Ascorbate | | | 0.02 | | | 0.02 | |
| pH | 3.03 | 2.5 | 4.46 | 4.16 | 2.56 | 3.02 | 3 |

### Example 6

### Stability Testing of Naloxone Formulations

The formulations listed in Table 11 were subjected to stability testing at 40° C. and 55° C.±2° C. under 75%±5% relative humidity for eight weeks. The stability data was collected at zero, one, two, three, four, at 55° C. and at zero, four weeks at 40° C. Assay and impurities were detected using high performance liquid chromatography with an ultraviolet detector. The assay was performed at 288 nm and indicated as a % of initial concentration. For all impurities, analysis was performed at 240 nm and expressed as a % area. Amounts of particular impurities are listed in Tables 12A to 12G and 13A to 13C as a percentage of the area of each formulation along with amount of total impurities. “BQL” refers to “Below Quantifiable Limit” and “ND” refers to “Not Detected.” “Ppm” refers to parts per million.

### Tables 12A to 12G. Stability Data for Sublingual Naloxone Spray Formulations Stored at 55° C.

### 12A. Stability of Control Stored at 55° C.

| Naloxone | RRT | T = 0 | 1 Week 55° C. |
|---|---|---|---|
| Assay (%) | | 100 | 101.48 |
| Impurity C | 0.66 | ND | ND |
| Impurity A | 0.83 | 0.15 | 0.15 |
| Impurity F | 0.93 | ND | ND |
| Impurity D | 1.14 | ND | ND |
| Impurity E | 3.20 | 0.07 | 0.62 |
| Impurity B | 3.40 | ND | ND |
| Unknown | 0.27 | ND | ND |
| Impurities | 0.33 | ND | BQL |
| | 0.36 | ND | ND |
| | 0.40 | ND | ND |
| | 0.49 | ND | 0.07 |
| | 0.56 | ND | ND |
| | 0.59 | ND | 0.12 |
| | 3.52 | ND | ND |
| Total | | 0.22% | 0.96% |
| Impurities | | | |

| | | | | | | 4 |
|---|---|---|---|---|---|---|
| Naloxone | RRT | T = 0 | 1 Week | 2 Weeks | 3 Weeks | Weeks |
| pH | | 4.469 | 4.21 | 4.239 | 4.02 | 4.224 |
| Assay (%) | | 100 | 99.6 | 101.48 | 98.07 | 98.00 |
| Impurity C | 0.66 | ND | BQL | BQL | BQL | BQL |
| Impurity A | 0.83 | BQL | 0.09 | 0.28 | 0.27 | 0.18% |
| Impurity F | 0.93 | ND | ND | ND | ND | ND |
| Impurity D | 1.14 | ND | ND | ND | ND | ND |
| Impurity E | 3.20 | ND | ND | ND | ND | ND |
| Impurity B | 3.40 | ND | ND | ND | ND | ND |
| Unknown | 0.33 | ND | BQL | 0.07 | 0.1 | 0.13 |
| Impurities | 0.36 | ND | BQL | ND | ND | ND |
| | 0.40 | ND | ND | ND | ND | ND |
| | 0.49 | ND | ND | 0.05 | 0.07 | 0.07% |
| | 0.56 | ND | 0.08 | 0.08 | 0.09 | 0.08 |
| | 0.59 | ND | 0.07 | 0.12 | 0.14 | 0.14 |
| | 3.90 | ND | 0.09 | 0.15 | 0.13 | 0.14 |
| Total | | 0.00% | 0.33% | 0.82% | 0.80% | 0.74% |
| Impurities | | | | | | |

### 12C. Stability of Form. #3AF (Edetate Disodium Dihydrate) Stored at 55° C.

| | | | | | 3 | 4 |
|---|---|---|---|---|---|---|
| Naloxone | RRT | T = 0 | 1 Weeks | 2 Weeks | Weeks | Weeks |
| pH | | 4.16 | 4.23 | 4.168 | 3.94 | 4.33 |
| Assay (%) | | 100 | 100.5 | 100.7 | 98.03 | 98.63 |
| Impurity | 0.66 | ND | ND | ND | ND | ND |
| C | | | | | | |
| Impurity | 0.83 | BQL | BQL | 0.21 | 0.18 | 0.07 |
| A | | | | | | |
| Impurity F | 0.93 | ND | ND | ND | ND | ND |
| Impurity | 1.14 | ND | ND | ND | ND | ND |
| D | | | | | | |
| Impurity E | 3.20 | 0.13 | 0.11 | 0.09 | 0.09 | 0.08 |
| Impurity | 3.40 | ND | ND | ND | ND | ND |
| B | | | | | | |
| Unknown | 0.33 | ND | BQL | 0.11 | 0.12 | 0.18 |
| Impurities | 0.36 | ND | BQL | BQL | BQL | BQL |
| | 0.40 | ND | ND | ND | ND | ND |
| | 0.49 | ND | ND | 0.09 | 0.1 | 0.11% |
| | 0.56 | ND | ND | ND | ND | ND |
| | 0.59 | ND | 0.12 | 0.11 | 0.14 | 0.15 |
| | 3.67 | ND | ND | ND | ND | 0.07 |
| | 3.90 | ND | 0.08 | 0.14 | 0.13 | 0.13 |
| Total | | 0.13% | 0.31% | 0.72% | 0.76% | 0.79% |
| Impuri- | | | | | | |
| ties | | | | | | |

### 12D. Stability of Form. #4AF (Edetate Disodium Dihydrate and L-Cysteine Hydrochloride) Stored at 55° C.

| | | | | | 3 | 4 |
|---|---|---|---|---|---|---|
| Naloxone | RRT | T = 0 | 1 Week | 2 Weeks | Weeks | Weeks |
| pH | | 2.56 | 2.5 | 2.44 | 2.38 | 2.413 |
| Assay (%) | | 100 | 98.5 | 100.03 | 97.87 | 98.59 |
| Impurity C | 0.66 | ND | ND | 0.13 | 0.11 | 0.09% |
| Impurity A | 0.83 | BQL | ND | 0.22 | 0.29 | 0.17% |
| Impurity F | 0.93 | ND | ND | ND | ND | ND |
| Impurity D | 1.14 | ND | ND | ND | ND | ND |
| Impurity E | 3.20 | ND | ND | ND | ND | ND |
| Impurity B | 3.40 | ND | ND | ND | ND | ND |
| Unknown | 0.33 | ND | ND | ND | ND | ND |
| Impurities | 0.36 | ND | ND | ND | ND | ND |
| | 0.40 | ND | ND | ND | ND | ND |
| | 0.49 | ND | ND | ND | ND | ND |
| | 0.56 | ND | ND | 0.05 | 0.07 | 0.06 |
| | 0.59 | ND | ND | ND | ND | ND |
| | 3.52 | ND | ND | ND | ND | ND |
| Total | | 0.00% | 0.00% | 0.40% | 0.47% | 0.32% |
| Impurities | | | | | | |

| | | | | |
|---|---|---|---|---|
| Naloxone | RRT | T = 0 | 1 Week | 2 Weeks |

| | | | | |
|---|---|---|---|---|
| pH | | NP | NP | 3.185 |
| Assay (%) | | 100 | 98.37 | 98.12 |
| Impurity C | 0.66 | ND | BQL | BQL |
| Impurity A | 0.83 | 0.15 | 0.18 | 0.11 |
| Impurity F | 0.93 | ND | ND | ND |
| Impurity D | 1.14 | ND | ND | ND |
| Impurity E | 3.20 | 0.07 | 0.16 | 0.12 |
| Impurity B | 3.40 | ND | ND | ND |
| Unknown | 0.27 | ND | ND | ND |
| Impurities | 0.33 | ND | ND | ND |
| | 0.36 | ND | ND | ND |
| | 0.40 | ND | ND | ND |
| | 0.49 | ND | BQL | BQL |
| | 0.56 | ND | ND | BQL |
| | 0.59 | ND | ND | ND |
| | 3.52 | ND | ND | ND |
| Total Impurities | | 0.22% | 0.34% | 0.23% |

### 12F. Stability of Form. #6AF (Edetate Disodium Dihydrate) Stored at 55° C.

| Naloxone | RRT | T = 0 | 1 Week | 2 Weeks | 3 Weeks | 4 Weeks |
|---|---|---|---|---|---|---|
| pH | | 3.013 | 3.443 | 3.132 | 3.241 | 3.21 |
| Assay (%) | | 100.00% | 98.34% | 98.36% | 98.34% | 100.07% |
| Impurity C | 0.66 | 0.10% | 0.10% | 0.21% | 0.13% | 0.13% |
| Impurity A | 0.83 | BQL | BQL | BQL | BQL | BQL |
| Impurity F | 0.93 | ND | ND | ND | ND | ND |
| Impurity D | 1.14 | ND | 0.83 ppm | 1.79 ppm | 1.74 ppm | ND |
| Impurity E | 3.20 | 0.15% | 0.15% | 0.15% | 0.17% | 0.17% |
| Impurity B | 3.40 | ND | ND | ND | ND | ND |
| Unknown | 0.27 | ND | ND | ND | ND | ND |
| Impurities | 0.33 | ND | ND | ND | ND | ND |
| | 0.36 | ND | ND | ND | ND | ND |
| | 0.40 | ND | ND | ND | ND | ND |
| | 0.49 | ND | BQL | 0.06% | 0.06% | 0.12% |
| | 0.56 | ND | ND | ND | ND | ND |
| | 0.59 | ND | ND | ND | ND | ND |
| | 0.77 | ND | ND | BQL | BQL | 0.05% |
| | 3.90 | ND | ND | ND | ND | ND |
| | 4.62 | ND | ND | BQL | BQL | ND |
| Total Impurities | | 0.25% | 0.25% | 0.42% | 0.36% | 0.47% |

### 12G. Stability of Form. #1AF (Edetate Disodium Dihydrate) Stored at 55° C.

| Naloxone | RRT | T = 0 | 1 Week | 2 Weeks | 3 Weeks | 4 Weeks |
|---|---|---|---|---|---|---|
| pH | | 2.505 | 2.907 | 2.581 | 2.616 | 2.62 |
| Assay (%) | | 100.00% | 107.80% | 100.51% | 100.17% | 102.39% |
| Impurity C | 0.66 | 0.10% | 0.10% | 0.10% | 0.10% | 0.09% |
| Impurity A | 0.83 | BQL | BQL | BQL | BQL | BQL |
| Impurity F | 0.93 | ND | ND | ND | ND | ND |
| Impurity D | 1.14 | NP | 0.95 ppm | 1.25 ppm | 1.59 ppm | ND |
| Impurity E | 3.20 | 0.15% | 0.14% | 0.15% | 0.16% | 0.18% |
| Impurity B | 3.40 | ND | ND | ND | ND | ND |
| Unknown | 0.06 | 0.13% | 0.13% | 0.13% | 0.13% | ND |
| Impurities | 0.27 | ND | ND | ND | ND | ND |
| | 0.33 | ND | ND | ND | ND | ND |
| | 0.36 | ND | ND | ND | ND | ND |
| | 0.40 | ND | ND | ND | ND | ND |
| | 0.47 | ND | ND | ND | ND | ND |
| | 0.49 | ND | BQL | 0.08% | 0.06% | 0.05% |
| | 0.56 | ND | ND | ND | ND | ND |
| | 0.77 | ND | ND | ND | BQL | BQL |
| | 3.61 | ND | ND | ND | BQL | ND |
| | 3.90 | ND | ND | ND | ND | ND |
| | 4.63 | ND | ND | ND | BQL | ND |
| Total Impurities | | 0.38% | 0.37% | 0.46% | 0.45% | 0.32% |

Sublingual naloxone formulations of the present invention contained less than 0.8% of total impurities after four weeks at 55° C. This is a stark contrast to the control formulation which contained 0.96% impurities after 1 week at 55° C. Specifically, the formulations which contained sodium ascorbate or edetate disodium dihydrate exhibited lower impurities after four weeks. Additionally, the formulations with a pH of about 2 to about 3 which contained edetate disodium dihydrate were very stable.

### Tables 13A to 13C. Stability Data for Sublingual Naloxone Spray Formulations Stored at 40° C. Under 75% Relative Humidity

### 13A. Stability of Form. #2AF (with Sod. Ascorbate & Edetate Disodium Dihydrate) Stored at 40° C. Under 75% Relative Humidity

| Naloxone | RRT | T = 0 | 4 Weeks |
|---|---|---|---|
| pH | | 4.469 | 4.394 |
| Assay (%) | | 100 | 98.12 |
| Impurity C | 0.66 | ND | 0.06 |
| Impurity A | 0.83 | BQL | 0.12 |
| Impurity F | 0.93 | ND | ND |
| Impurity D | 1.14 | ND | ND |
| Impurity E | 3.20 | ND | ND |
| Impurity B | 3.40 | ND | ND |
| Unknown | 0.33 | ND | BQL |
| Impurities | 0.36 | ND | ND |
| | 0.40 | ND | ND |
| | 0.49 | ND | 0.06 |
| | 0.56 | ND | ND |
| | 0.59 | ND | 0.06 |
| | 3.90 | ND | 0.14 |
| Total Impurities | | 0.00% | 0.44% |

### 13B. Stability of Form. #3AF (Edetate Disodium Dihydrate) Stored at 40° C. Under 75% Relative Humidity

| Naloxone | RRT | T = 0 | 4 Weeks |
|---|---|---|---|
| pH | | 4.16 | 4.596 |
| Assay (%) | | 100 | 99.69 |
| Impurity C | 0.66 | ND | ND |
| Impurity A | 0.83 | BQL | BQL |
| Impurity F | 0.93 | ND | ND |
| Impurity D | 1.14 | ND | ND |
| Impurity E | 3.20 | 0.13 | ND |
| Impurity B | 3.40 | ND | ND |
| Unknown | 0.33 | ND | BQL |
| Impurities | 0.36 | ND | ND |
| | 0.40 | ND | ND |
| | 0.49 | ND | BQL |
| | 0.56 | ND | ND |
| | 0.59 | ND | 0.11 |
| | 3.67 | ND | ND |
| | 3.90 | ND | 0.11 |
| Total | | 0.13% | 0.22% |
| Impurities | | | |

### 13C. Stability of Form. #4AF (Edetate Disodium Dihydrate and L-Cysteine hydrochloride) Stored at 40° C. under 75% Relative Humidity

| Naloxone | RRT | T = 0 | 4 Weeks |
|---|---|---|---|
| pH | | 2.56 | 2.502 |
| Assay (%) | | 100 | 97.08 |
| Impurity C | 0.66 | ND | ND |
| Impurity A | 0.83 | BQL | BQL |
| Impurity F | 0.93 | ND | ND |
| Impurity D | 1.14 | ND | ND |
| Impurity E | 3.20 | ND | ND |
| Impurity B | 3.40 | ND | ND |
| Unknown | 0.33 | ND | ND |
| Impurities | 0.36 | ND | ND |
| | 0.40 | ND | ND |
| | 0.49 | ND | ND |
| | 0.56 | ND | ND |
| | 0.59 | ND | ND |
| | 3.52 | ND | ND |
| Total | | 0.00% | 0.00% |
| Impurities | | | |

The naloxone formulations of the present invention contained less than 0.45% of total impurities after four weeks at 40° C.

### Example 7

### Freeze/Thaw Testing

In order to further determine the stability of Formulations #1AF and #6AF, the formulations were subjected to standard freeze/thaw stability testing. The results are below in “Table 14. Stability of Formulations #1AF and #6AF to Freeze/Thaw Testing.”

TABLE 14
Stability of Formulations #1AF and #6AF to Freeze/Thaw Testing

| Formulation | | | | | | | | |
|---|---|---|---|---|---|---|---|---|
| #1AF to | Drug | | | Cycle 1, | | Cycle 2, | | Cycle 3, |
| #6AF | Substance | t = 0 | Cycle 1, −20° C. | 25° C. | Cycle 2, −20° C. | 25° C. | Cycle 3, −20° C. | 25° C. |
| Date | | Mar. 12, 2015 | Mar. 16, 2015 | Mar. 18, 2015 | Mar. 20, 2015 | Mar. 22, 2015 | Mar. 24, 2015 | Mar. 26, 2015 |
| Observed: | | | | | | | | |
| Physical | clear | clear | clear | clear | clear | Clear | clear | clear |
| appearance | | | | | | | | |
| Color | colorless | colorless | colorless | colorless | colorless | colorless | colorless | Colorless |

The naloxone formulations #1AF to #6AF were clear and colorless after several cycles of freezing and thawing. This study further demonstrates the stability of the formulations.

### Example 8

### Droplet Testing

In order to determine the spray profile of Formulation #1AF, it was subjected to standardized droplet testing. As previously explained, the optimal particle size for sublingual spray droplets is from 20 to about 200 microns in diameter. It is desirable for the formulation to have droplet sizes near 20 because this increases the surface area and increased surface area exposure is one factor that contributes to a high bioavailability. Sublingual formulations should be able to maintain a consistent droplet size throughout its shelf life.

Droplet analysis was conducted using standard laser analysis procedures known by those of skill in the art. Droplet size distribution (Dv10, Dv50, Dv90, and Span were tested at two distances, 3 cm and 6 cm). Dv10 refers to droplet size for which 10% of the total volume is obtained; Dv50 refers to droplet size for which 50% of the total volume is obtained; Dv90 refers to droplet size for which 90% of the total volume is obtained; Span refers to distribution span (Dv90-Dv10)/Dv50; % RSD refers to the percent relative standard deviation. The results of these tests can be seen below in Tables 15 to 20. Applicant found during testing that formulations of the present invention yielded desirable droplet sizes for sublingual administration. The testing also revealed that the formulation dose remains consistent when administered with a spray pump.

TABLE 15
Spray Profile of Naloxone Spray Formulation #1AF,
Particle Size at 3 cm

Particle Size

| Formulation #1AF | | DV(10) | DV(50) | DV(90) | % <10μ | Span |
|---|---|---|---|---|---|---|
| 3 cm | Actuation 1 | 13.16 | 26.23 | 63.21 | 2.792 | 1.908 |
| | Actuation 2 | 11.52 | 27 | 90.85 | 6.547 | 2.939 |
| | Actuation 3 | 12.95 | 28.39 | 144 | 3.505 | 4.615 |
| | Average | 12.54 | 27.21 | 99.4 | 4.281 | 3.15 |

TABLE 16
Spray Profile of Naloxone Spray Formulation #1AF,
Particle Size at 6 cm

Particle Size

| Formulation #1AF | | DV(10) | DV(50) | DV(90) | % <10μ | Span |
|---|---|---|---|---|---|---|
| 6 cm | Actuation 1 | 20.18 | 32.51 | 53.9 | 1.198 | 1.037 |
| | Actuation 2 | 18.02 | 31.45 | 58.48 | 0.024 | 1.286 |
| | Actuation 3 | 16.81 | 33.44 | 77.92 | 1.799 | 1.828 |
| | Average | 18.34 | 32.47 | 63.4 | 1.007 | 1.38 |

TABLE 17
Spray Profile of Naloxone Spray Formulation
#1AF, Spray Pattern at 3 cm

| | | Spray Pattern | | |
|---|---|---|---|---|
| Formulation #1AF | | Dmin (mm) | Dmax (mm) | Ovality Ratio |
| 3 cm | Actuation 1 | 26.5 | 41.3 | 1.557 |
| | Actuation 2 | 24.8 | 43.5 | 1.751 |
| | Actuation 3 | 29 | 40.6 | 1.402 |
| | Average | 26.8 | 41.8 | 1.570 |

TABLE 18
Spray Profile of Naloxone Spray Formulation
#1AF, Spray Pattern at 6 cm

| | | Spray Pattern | | |
|---|---|---|---|---|
| Formulation #1AF | | Dmin (mm) | Dmax (mm) | Ovality Ratio |
| 6 cm | Actuation 1 | 52.6 | 68.6 | 1.304 |
| | Actuation 2 | 40.3 | 61.4 | 1.524 |
| | Actuation 3 | 47.5 | 59.7 | 1.256 |
| | Average | 46.8 | 63.2 | 1.361 |

TABLE 19
Spray Profile of Naloxone Spray Formulation
#1AF, Plume geometry data at 3 cm

Plume Geometry

| | | |
|---|---|---|
| Formulation #1AF | Width (mm) | Angle (°) |

| | | | |
|---|---|---|---|
| 3 cm | Actuation 1 | 39.7 | 66.7 |
| | Actuation 2 | 37.7 | 64.3 |
| | Actuation 3 | 33.5 | 58 |
| | Average | 37.0 | 63.0 |

TABLE 20
Spray Profile of Naloxone Spray Formulation
#1AF, Plume geometry data at 6 cm

Plume Geometry

| | | |
|---|---|---|
| Formulation #1AF | Width (mm) | Angle (°) |

| | | | |
|---|---|---|---|
| 6 cm | Actuation 1 | 63 | 54.9 |
| | Actuation 2 | 67.1 | 58.3 |
| | Actuation 3 | 68 | 59 |
| | Average | 66.0 | 57.4 |

As can be seen in Tables 15 to 20, Formulation #1AF of the present invention provided excellent plume geometry and spray patterns.

### Example 9

### Preparation of Additional Naloxone Sublingual Formulations

In order to prepare naloxone sublingual formulations, the components as indicated in “Table 21. The Components of Formulations #8A, #9A, #7AF and #8AF” below were weighed. The components were mixed until a clear solution was formed.

Strawberry flavoring was used as the source of flavoring agent.

TABLE 21
The Components of Formulations #8A, #9A, #7AF and #8AF

| Formulation | Control | #8A | #9A | #7AF | #8AF |
|---|---|---|---|---|---|
| Naloxone | 2.44 | 10.419 | 10.265 | 4.4196 | 4.4196 |
| Water (USP) | 37.56 | 29.506 | 31.324 | 94.769 | 94.779 |
| Alcohol | 55 | 55 | 50 | | |
| Propylene Glycol | 5 | 5 | 5 | | |
| L-menthol | | 0.05 | 0.5 | | |
| BKC | | | 0.01 | 0.01 | |
| Sodium Chloride | | | | 0.8 | 0.8 |
| Flavoring agent | | | 0.08 | | |
| Edetate disodium | | 0.005 | 0.001 | 0.001 | 0.001 |
| dihydrate | | | | | |
| Sucralose | | | 0.8 | | |
| Caprylic Acid | | | 2 | | |
| Sodium Ascorbate | | 0.02 | 0.02 | | |

values = % w/w
pH - 4.5 ± 0.2

TABLE 22
Stability of Formulations # 9A and # 8A to Freeze/Thaw Testing

| | Drug | | Cycle 1, | Cycle 1, | Cycle 2, | Cycle 2, | Cycle 3, | Cycle 3, |
|---|---|---|---|---|---|---|---|---|
| Formulation | Substance | t = 0 | 20° C. | 40° C. | 20° C. | 40° C. | 20° C. | 40° C. |
| Date | | Jan. 12, | Jan. 14, 2016 | Jan. 16, 2016 | Jan. 18, 2016 | Jan. 20, 2016 | Jan. 22, 2016 | Mar. 24, 2015 |
| Observed: | | 2016 | | | | | | |
| Physical | clear | clear | clear | clear | clear | Clear | clear | clear |
| appearance | | | | | | | | |
| Color | colorless | colorless | colorless | colorless | colorless | colorless | colorless | Colorless |

The naloxone formulations #8A and #9A were clear and colorless after several cycles of freezing and thawing. This study further demonstrates the stability of the formulations.

### Example 10

### Droplet Testing

In order to determine the spray profile of Formulation #9A, it was subjected to standardized droplet testing. As previously explained, the optimal particle size for sublingual spray droplets is from 20 to about 200 microns in diameter. It is desirable for the formulation to have droplet sizes near 20 because this increases the surface area and increased surface area exposure is one factor that contributes to a high bioavailability. Sublingual formulations should be able to maintain a consistent droplet size throughout its shelf life.

Droplet analysis was conducted using standard laser analysis procedures known by those of skill in the art. Droplet size distribution (Dv10, Dv50, Dv90, and Span were tested at two distances, 3 cm and 6 cm). Dv10 refers to droplet size for which 10% of the total volume is obtained; Dv50 refers to droplet size for which 50% of the total volume is obtained; Dv90 refers to droplet size for which 90% of the total volume is obtained; Span refers to distribution span (Dv90−Dv10)/Dv50; % RSD refers to the percent relative standard deviation. The results of these tests can be seen below in Tables 23 to 28. Applicant found during testing that formulations of the present invention yielded desirable droplet sizes for sublingual administration. The testing also revealed that the formulation dose remains consistent when administered with a spray pump.

TABLE 23
Spray Profile of naloxone Spray Formulation #9A,
Particle Size at 3 cm

| | | Particle Size | | | | |
|---|---|---|---|---|---|---|
| Formulation #9A | | DV(10) | DV(50) | DV(90) | % <10μ | Span |
| 3 cm | Actuation 1 | 23.06 | 44.28 | 99.7 | 0 | 1.731 |
| | Actuation 2 | 22.08 | 44.34 | 104.4 | 0.661 | 1.856 |
| | Actuation 3 | 22.27 | 55.05 | 107.5 | 1.012 | 2.82 |
| | Average | 22.47 | 47.89 | 103.9 | 0.558 | 2.14 |

TABLE 24
Spray Profile of Naloxone Spray Formulation #9A,
Particle Size at 6 cm

| | | Particle Size | | | | |
|---|---|---|---|---|---|---|
| Formulation #9A | | DV(10) | DV(50) | DV(90) | % <10μ | Span |
| 6 cm | Actuation 1 | 28.54 | 51.29 | 91.87 | 2.113 | 1.235 |
| | Actuation 2 | 25.75 | 50.01 | 103.7 | 1.594 | 1.56 |
| | Actuation 3 | 31.99 | 51.77 | 85 | 0 | 1.024 |
| | Average | 28.76 | 51.02 | 93.5 | 1.236 | 1.27 |

TABLE 25
Spray Profile of Naloxone Spray Formulation
#9A, Spray Pattern at 3 cm

| | | Spray Pattern | | |
|---|---|---|---|---|
| Formulation #9A | | Dmin (mm) | Dmax (mm) | Ovality Ratio |
| 3 cm | Actuation 1 | 14.7 | 22.7 | 1.544 |
| | Actuation 2 | 14.4 | 21.8 | 1.517 |
| | Actuation 3 | 15.2 | 20.9 | 1.372 |
| | Average | 14.8 | 21.8 | 1.478 |

TABLE 26
Spray Profile of Naloxone Spray Formulation
#9A, Spray Pattern at 6 cm

| | | Spray Pattern | | |
|---|---|---|---|---|
| Formulation #9A | | Dmin (mm) | Dmax (mm) | Ovality Ratio |
| 6 cm | Actuation 1 | 23.5 | 30.4 | 1.291 |
| | Actuation 2 | 24.5 | 41.8 | 1.707 |
| | Actuation 3 | 20.5 | 32.7 | 1.597 |
| | Average | 22.8 | 35.0 | 1.532 |

TABLE 27
Spray Profile of Naloxone Spray Formulation
#9A, Plume geometry data at 3 cm

Plume Geometry

| | | |
|---|---|---|
| Formulation #9A | Width (mm) | Angle (°) |

| | | | |
|---|---|---|---|
| 3 cm | Actuation 1 | 22.5 | 39.9 |
| | Actuation 2 | 15.5 | 28.6 |
| | Actuation 3 | 25.7 | 44.3 |
| | Average | 21.2 | 37.6 |

TABLE 28
Spray Profile of Naloxone Spray Formulation
#9A, Plume geometry data at 6 cm

Plume Geometry

| | | |
|---|---|---|
| Formulation #9A | Width (mm) | Angle (°) |

| | | | |
|---|---|---|---|
| 6 cm | Actuation 1 | 26.4 | 24.3 |
| | Actuation 2 | 25 | 23.3 |
| | Actuation 3 | 37.6 | 33.2 |
| | Average | 29.7 | 26.9 |

As can be seen in Tables 23 to 28, Formulation #9A of the present invention provided excellent plume geometry and spray patterns.

### Example 11

### Pharmacokinetic Analysis

The naloxone formulations described in Example 9, Table 21 of the instant specification were used. For formulations #7AF and #8AF a 4 mg dose was administered. For formulations #8A and #9A a 16 mg dose was administered.

Pharmacokinetic and Bioavailability Analysis

Protocol was a single dose crossover study. Five healthy male Yucatan minipigs weighing approximately forty kilograms each were sublingually administered the formulations of Table 21. The minipigs were fasted overnight and through four hours post administration. Administration was followed by a one week washout period. Blood samples were taken prior to administration and 1, 3, 5, 7, 10, 15, 30 min, 1, 2, 4, 8 and 24 hours postdose. Blood samples were measured for naloxone concentrations via liquid chromatography-tandem mass spectrometry.

The following pharmacokinetic parameters were calculated: peak concentration in plasma (Cₘₐₓ) and area under the concentration-time curve from time-zero to the time of the last quantifiable concentration (AUC₀₋ₜ).

Results and Conclusions

Results of the pharmacokinetic and statistical analysis for the naloxone formulations in Table 21 of the present invention are shown in Table 29.

TABLE 29
Summary of pharmacokinetic parameters for naloxone after sublingual administration of single
doses of 4 mg and 16 mg of naloxone formulations to Yucatan minipigs under fasted conditions.

| Parameter* | #7AF | #8AF | #8A | #9A | #9A (repeat) |
|---|---|---|---|---|---|
| Cₘₐₓ (ng/mL) | 8.9 ± 2.2 | 6.8 ± 2.2 | 26.3 ± 1.8 | 86.4 ± 2.4 | 58.6 ± 2.8 |
| Conc. @ 1 min | NA | 0.1 | NA | 24.2 | NA |
| (ng/mL) | | | | | |
| Conc. @ 3 min | 0.2 | 0.3 | 5 | 68.5 | 12.9 |
| (ng/mL) | | | | | |
| Conc. @ 5 min | 1.4 | 1.9 | 6.3 | 62.7 | 36.9 |
| (ng/mL) | | | | | |
| Conc. @ 7 min | 1.6 | 2.6 | 9.1 | 28.8 | 50.5 |
| (ng/mL) | | | | | |
| AUC₍₀₋ₜ₎ | 746.2 ± 2.2 | 432.3 ± 2.0 | 2382.5 ± 2.0 | 3108.5 ± 2.2 | 3564.8 ± 2.8 |
| (ng*min/mL) | | | | | |
| AUC @ 15 min | 41.6 | 38 | 188.2 | 615.8 | 515.6 |
| (ng*min/mL) | | | | | |
| AUC @ 30 min | 151.8 | 106.8 | 504.4 | 987.4 | 1063.3 |
| (ng*min/mL) | | | | | |

*Geometric mean ± geometric standard deviation. Sample size is 5.

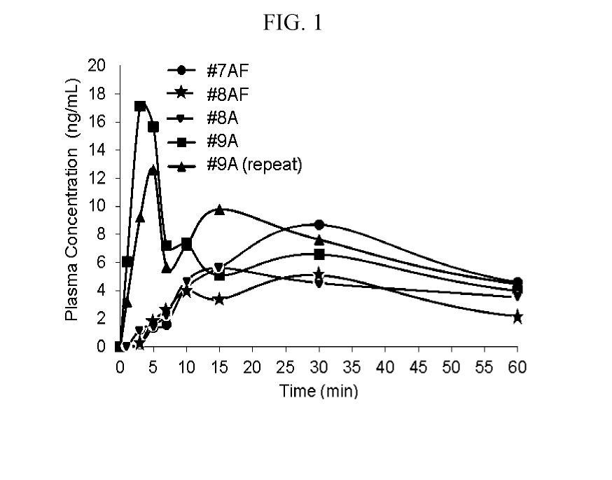

The peak mean naloxone concentration was significantly higher for formulation #9A and #8A over #8AF and #7AF. Additionally, the area under the concentration-time curve from time-zero to the time of the last quantifiable concentration was significantly higher for formulations #9A and #8A over #8AF and #7AF. To determine if this result was based on the four fold increase in the dose of naloxone in formulation #9A and #8A over #8AF and #7AF the geometric mean was normalized to 4 mg dose. See FIG. 1. A similar pattern remains even after normalization. Further, the peak mean naloxone concentration was significantly higher for formulation #9A, over #8A, which cannot be explained by the dosage as formulations #9A and #8A were each administered at 16 mg doses.

Additionally, formulation #9A reached about 80% of its peak mean naloxone concentration within 3 minutes of administration. In comparison, formulation #8A had reached only 35% of its peak mean naloxone concentration within 7 minutes, #8AF 38% in 7 minutes and #7AF 19% in 7 minutes. In a similar comparison formulation #9A reached 19% of its AUC₍₀₋ₜ₎ within 15 minutes of administration, #8A reached 7.9% in 15 minutes, #8AF reached 8.8% in 15 minutes and #7AF reached 5.6% in 15 minutes.

Administration of naloxone in formulations with cosolvents resulted in superior bioavailability. Compare formulation #9A and #8A to #8AF and #7AF. Further, the addition of permeation enhancers such as caprylic acid and BKC resulted in further increase in bioavailability. Compare formulations #9A to #8A and #7AF to #8AF.

### Example 12

### Stability Testing of Additional Naloxone Formulations

Formulation #9A from Table 21 above was subjected to stability testing at 25° C./60% RH±5%, 40° C./75%±5% relative humidity and 55° C.±2° C. four weeks. The stability data was collected at predetermined time points. Assay and impurities were detected using high performance liquid chromatography with an ultraviolet detector. The assay was performed at 288 nm and indicated as a % of initial concentration. For all impurities, analysis was performed at 240 nm and expressed as a % area. Amounts of particular impurities are listed in Tables 30 A to C as a percentage of the area of each formulation along with amount of total impurities.

TABLE 30A
Stability Data for the Formulation #9A Stored at 25°
C. ± 2° C./60% ± 5% RH

| Naloxone | RRT | T = 0 | 4 Weeks |
|---|---|---|---|
| Physical Appearance | — | Clear, colorless | Clear, light yellow |
| (Clarity, Color) | | | |
| pH | — | 4.5 | 4.4 |
| Assay (%) | — | 100 | 98.7 |
| Impurity C | 0.66 | 0.02 | 0.01 |
| Impurity A | 0.83 | ND | 0.02 |
| Impurity F | 0.93 | ND | ND |
| Impurity D | 1.14 | ND | 2.66 ppm |
| Impurity E | 3.08 | 0.05 | 0.05 |
| Impurity B | 3.40 | ND | ND |
| Unknown | 0.27 | ND | ND |
| Impurities | 0.33 | ND | 0.01 |
| | 0.36 | ND | ND |
| | 0.40 | ND | ND |
| | 0.49 | ND | ND |
| | 0.56 | ND | ND |
| | 0.58 | ND | ND |
| | 0.77 | ND | 0.02 |
| | 2.34 | ND | 0.01 |
| | 3.30 | ND | 0.05 |
| Total | — | 0.07% | 0.17% |
| Impurities | | | |

ND = Not Detected
ppm = parts per million

TABLE 30B
Stability Data for the Formulation #9A Stored at 40°
C. ± 2° C./75% ± 5% RH

| Naloxone | RRT | T = 0 | 4 Weeks |
|---|---|---|---|
| Physical Appearance | — | Clear, colorless | Clear, light yellow |
| (Clarity, Color) | | | |
| pH | — | 4.46 | 4.42 |
| Assay (%) | — | 100 | 99.09 |
| Impurity C | 0.66 | 0.02 | 0.01 |
| Impurity A | 0.83 | ND | 0.05 |
| Impurity F | 0.93 | ND | ND |
| Impurity D | 1.14 | ND | 2.24 ppm |
| Impurity E | 3.08 | 0.05 | 0.04 |
| Impurity B | 3.40 | ND | ND |
| Unknown | 0.33 | ND | 0.01 |
| Impurities | 0.36 | ND | ND |
| | 0.40 | ND | ND |
| | 0.49 | ND | 0.01 |
| | 0.56 | ND | ND |
| | 0.58 | ND | 0.04 |
| | 0.77 | ND | 0.02 |
| | 1.76 | ND | 0.01 |
| | 2.34 | ND | 0.01 |
| | 3.30 | ND | 0.02 |
| Total | — | 0.07% | 0.22% |
| Impurities | | | |

ND = Not Detected
ppm = parts per million

TABLE 30C
Stability Data for the Formulation # 9A Stored at 55° C. ± 2° C.

| Naloxone | RRT | T = 0 | 2 Weeks | 4 Weeks |
|---|---|---|---|---|
| Physical Appearance | | Clear, | Clear, | Clear, |
| (Clarity, Color) | | colorless | light yellow | yellow |
| pH | | 4.5 | 4.3 | 4.23 |
| Assay (%) | | 100 | 99.31 | 97.28 |
| Impurity C | 0.66 | ND | 0.01 | 0.02 |
| Impurity A | 0.83 | 0.02 | 0.14 | 0.19 |
| Impurity F | 0.93 | ND | ND | ND |
| Impurity D | 1.14 | ND | ND | 9.36 ppm |
| Impurity E | 3.08 | 0.05 | 0.07 | 0.11 |
| Impurity B | 3.40 | ND | ND | ND |
| Unknown | 0.27 | ND | ND | ND |
| Impurities | 0.33 | ND | 0.03 | 0.05 |
| | 0.36 | ND | 0.04 | 0.07 |
| | 0.40 | ND | ND | ND |
| | 0.49 | ND | 0.08 | 0.08 |
| | 0.53 | ND | ND | 0.01 |
| | 0.58 | ND | 0.09 | 0.09 |
| | 0.59 | ND | 0.04 | 0.08 |
| | 0.70 | ND | ND | 0.01 |
| | 1.75 | ND | 0.04 | 0.06 |
| | 1.90 | ND | ND | 0.01 |
| | 1.96 | ND | ND | 0.01 |
| | 2.33 | ND | ND | 0.03 |
| | 3.15 | ND | ND | 0.01 |
| | 3.22 | ND | ND | 0.04 |
| Total | | 0.07% | 0.54% | 0.87% |
| Impurities | | | | |

ND = Not Detected
ppm = parts per million

The data suggest that formulation #9A demonstrates satisfactory stability with no significant increase in individual or total impurities. Based upon these results, the formulation containing 0.02% w/w of sodium ascorbate as an antioxidant and 0.001% edetate disodium dihydrate as a chelating agent is chemically stable.

1. A storage stable sublingual spray formulation comprising an effective amount of naloxone, a pharmaceutically acceptable salt thereof, or a derivative thereof, water, a cosolvent and a permeation enhancer.

2. The sublingual spray formulation of claim 1, wherein the pH is from about 2 to about 7.

3. The sublingual spray formulation of claim 1, wherein:
the cosolvent is selected from the group consisting of an alcohol, a glycol, and a combination thereof; and
the permeation enhancer is selected from the group consisting of menthol, limonene, carvone, methyl chitosan, caprylic acid, pelargonic acid, capric acid, undecylenic acid, lauric acid, myristic acid, palmitic acid, oleic acid, stearic acid, linolenic acid, arachidonic acid, polysorbates, edetate disodium dihydrate, benzalkonium chloride (BKC), cetylpyridinium chloride, sodium lauryl sulfate, citric acid, sodium desoxycholate, sodium deoxyglycolate, glyceryl oleate, glyceryl monosterate, sodium hydroxybenzoyal amino caprylate, sodium caprate, dodecyl dimethyl aminopropionate, L-lysine, sodium glycocholate, citric acid, peppermint oil and a combination thereof.

4. The sublingual spray formulation of claim 3, wherein the alcohol is ethanol (dehydrated alcohol) and the glycol is propylene glycol.

5. A storage stable sublingual spray formulation comprising:
from about 0.01% to about 20% w/w naloxone, a pharmaceutically acceptable salt or a derivative thereof;
from about 10% to about 99% w/w water;
from about 5% to about 90% w/w of a cosolvent; and
from about 0.001% to 10.0% of a permeation enhancer,
wherein the % w/w is of the total formulation.

6. The sublingual spray formulation of claim 5, wherein the permeation enhancer is selected from the group consisting of menthol, limonene, carvone, methyl chitosan, caprylic acid, pelargonic acid, capric acid, undecylenic acid, lauric acid, myristic acid, palmitic acid, oleic acid, stearic acid, linolenic acid, arachidonic acid, polysorbates, edetate disodium dihydrate, benzalkonium chloride (BKC), cetylpyridinium chloride, sodium lauryl sulfate, citric acid, sodium desoxycholate, sodium deoxyglycolate, glyceryl oleate, glyceryl monosterate, sodium hydroxybenzoyal amino caprylate, sodium caprate, dodecyl dimethyl aminopropionate, L-lysine, sodium glycocholate, citric acid, peppermint oil and a combination thereof.

7. The sublingual spray formulation of claim 6, wherein the permeation enhancer is selected from the group consisting of menthol, benzalkonium chloride, caprylic acid, edetate disodium dihydrate and a combination thereof.

8. The sublingual spray formulation of claim 5, further comprising an antioxidant selected from the group consisting of butylated hydroxyanisole (BHA), butylated hydroxytoluene (BHT), methionine, sodium ascorbate, sodium thiosulfate, thioglycerol, ascorbic acid, propyl gallate, dL-alpha-tocopherol, sodium sulfite, sodium metabisulfite, sodium bisulfate, cysteine hydrochloride, glutathione and a combination thereof.

9. A method of treating opioid dependence comprising administering the sublingual spray formulation of claim 1 to a patient in need thereof.

10. A method of treating opioid overdose comprising administering the sublingual spray formulation of claim 1 to a patient in need thereof.

11. A method of treating congenital insensitivity to pain with anhidrosis comprising administering the sublingual spray formulation of claim 1 to a patient in need thereof.

12. A storage stable sublingual spray formulation comprising an effective amount of naloxone, a pharmaceutically acceptable salt or a derivative thereof, water, and a permeation enhancer, wherein the formulation does not contain alcohol.

13. The sublingual spray formulation of claim 12, wherein the pH is from about 2 to about 7.

14. The sublingual spray formulation of claim 12, wherein the permeation enhancer is selected from the group consisting of polysorbates, edetate disodium dihydrate, benzalkonium chloride (BKC), cetylpyridinium chloride, sodium lauryl sulfate, citric acid, sodium desoxycholate, sodium deoxyglycolate, glyceryl oleate, glyceryl monosterate, L-lysine, sodium glycocholate, sodium taurocholate, citric acid, and a combination thereof.

15. The sublingual spray formulation of claim 14, further comprising a chelating agent.

16. The sublingual spray formulation of claim 15, wherein the chelating agent is edetate disodium dihydrate.

17. The sublingual spray formulation of claim 16, wherein:
naloxone, a pharmaceutically acceptable salt or a derivative thereof is at a concentration from about 0.01% to about 20% w/w;
water is at a concentration from about 20% to about 99% w/w;
the permeation enhancer is at a concentration from about 0.001% to about 10% w/w; and
edetate disodium dihydrate is at a concentration from about 0.0001% to 0.5% w/w, wherein the % w/w is of the total formulation.

18. The sublingual spray formulation of claim 17, wherein:
naloxone, a pharmaceutically acceptable salt or a derivative thereof is at a concentration from about 1% to about 12% w/w;
water is at a concentration from about 30% to about 98% w/w;
the permeation enhancer is at a concentration from about 0.001% to about 2.5% w/w; and
edetate disodium dihydrate is at a concentration from about 0.001% to 0.5% w/w.

19. A method of treating opioid dependence comprising administering the sublingual spray formulation of claim 12 to a patient in need thereof.

20. A method of treating opioid overdose comprising administering the sublingual spray formulation of claim 12 to a patient in need thereof.

21. A method of treating congenital insensitivity to pain with anhidrosis comprising administering the sublingual spray formulation of claim 12 to a patient in need thereof.
